(19) United States
(12) Patent Application Publication
(10) Pub. No.: US 2026/0112460 A1
(43) Pub. Date: Apr. 23, 2026

(54) PROCESSES, MACHINES, AND ARTICLES OF MANUFACTURE RELATED TO PREDICTING EFFECTS OF COMBINATIONS OF ITEMS

(71) Applicant: Flagship Pioneering Innovations VI, LLC, Cambridge, MA (US)

(72) Inventors: Hok Hei Tam, Newton, MA (US); Varun Shivashankar, Waltham, MA (US); Nathan Edward Sanders, North Andover, MA (US); Changchang Liu, Cambridge, MA (US); David Barry Kolesky, Arlington, MA (US); Duminda Sampath Ranasinghe, Cambridge, MA (US); Fernanda Menezes Cerqueira, Winthrop, MA (US)

(21) Appl. No.: 18/993,327

(22) PCT Filed: Jul. 11, 2023
(86) PCT No.: PCT/US2023/069973

§ 371 Date: Jan. 10, 2025

Related U.S. Application Data
(60) Provisional application No. 63/368,161, filed on Jul. 12, 2022.
(60) Provisional application No. 63/375,730, filed on Sep. 15, 2022.

(51) Int. Cl. G16C 20/30; G16C 20/70

(52) U.S. Cl. CPC G16C 20/30; G16C 20/70

(57) ABSTRACT

A computational model outputs, and allows supervision based on, a set of values representing a response manifold representing a combined effect of two items together. The set of values can be a matrix of values sampling the response surface, or set of parameters of a function representing the response surface A matrix of known values for researched items can be used as supervisory information during training, either directly or indirectly by conversion into parameters representing the response surface. A matrix of values representing the combined effect of researched items may be incomplete. This information can be processed to complete the set of values, or to generate a parameterized representation of the response surface. The computational model can be constructed to include parameters representing uncertainty associated with the measurements of combined effects. Multiple computational models can be used. A form of ensembling can be used to combine the outputs of multiple models.

### BACKGROUND

Machine learning generally involves using data about one set of items for which a property is known, such as classifications for the items, to train a computational model that in turn can make predictions about what that property should be for other items, for which that property is not known. While there is a wide range of possible applications of this general concept of machine learning, practical applications can be hard to implement for many reasons.

### SUMMARY

This Summary introduces a selection of concepts in simplified form that are described further below in the Detailed Description. This Summary neither identifies key or essential features, nor limits the scope, of the claimed subject matter.

Drug combinations have been shown to be an effective strategy for therapy for many different health condition. However, drug response, compound-protein interaction, and combinatoric effect prediction, require diverse data to supervise training across several dimensions: chemical space, chemical interactions (combinations), dose, biological models (e.g. cell lines), and biological responses (e.g. cytotoxicity). This high dimensionality makes dense experimental sampling labor-intensive and expensive.

Machine learning techniques can be used to build a computer system that can predict properties of items, such as drug dose response or the effect of drug combinations. To do so, the computer system has access to data representing a set of researched items for which a property is known. The property which a researched item has is one from among a plurality of types of properties. The computer system also has access to data representing potential candidate items. For each potential candidate item, respective information is not known for at least one property among the plurality of types of properties. The computer system applies machine learning techniques to train a computational model using the data representing the researched items and their known properties, for a plurality of types of properties. The computer system applies the trained computational model to the data representing the potential candidate items. In response, the trained computational model outputs one or more predictions about whether the potential candidate items are likely to have a property from among the plurality of types of properties that the computational model is trained to predict.

In some practical applications, it can be useful to consider combinations of items and effects produced by such combinations of items, especially in different quantities of those items. In such applications, the property of a researched item which is known can be a combined effect of a first item and a second item together. In some implementations, a computer system may represent the researched item as a first item, and its property is its combined effect with the second item. In some implementations, a computer system may represent the researched item as the combination of the first item and the second item, and the property of the researched item is their combined effect.

It is possible that this combined effect may be known for a plurality of different combinations of quantities of the first item and second item together. For example, the combined effect may be known for the first item in a first quantity together with a quantity of the second item and the combined effect may be known for the first item in a second quantity together with the quantity of the second item. For example, the combined effect also may be known for the first item in the first quantity together with a different quantity of the second item and the combined effect may be known for the first item in the second quantity with the different quantity of the second item.

Generally, a combined effect can be represented as a response manifold over a domain of two or more inputs. With two inputs, the combined effect can be represented as a response surface. Data representing the combined effect can be a matrix of values representing a mapping of different quantities of different items to the respective combined effects of those items in those different quantities. In some implementations, such a matrix may be incomplete based on known information about researched items. For example, information may be available for a combined effect for a pair of items in some quantities, but not in other quantities. In some implementations, the data representing the combined effect can be a set of values for a set of parameters of a model that describes the response manifold.

To train a computational model, a selected subset of the researched items is used, called a training set. Researched items in the selected subset have information characterizing a known property of the researched item. The property which a researched item has can be one from among a plurality of types of properties.

The property of a researched item can be a combined effect of at least a first item and a second item. The combined effect may be known for the first item and the second item together in a plurality of distinct combinations of different quantities. The selected researched items can be those for which some information about the combined effects are known.

A trained computational model is applied to a selected subset of a plurality of potential candidate items, called a target set, to generate a respective result set. The result set comprises data representative of a set of predicted candidate items from among the plurality of potential candidate items. The trained computational model predicts, based on the selected subset of potential candidate items, whether the predicted candidate items are likely to have one or more types of properties. The result set can include, for each predicted candidate item, a respective prediction value for the predicted candidate item for a type of property.

The property of a predicted candidate item can be a predicted combined effect of at least a first item and a second item. The trained computational model predicts whether each predicted candidate item is likely to have, in a respective combination including the predicted candidate item with another item, a combined effect. The trained computational model can provide a respective prediction value indicative of a predicted combined effect for the respective combination including the predicted candidate item.

Many such relationships are described by nonlinear sigmoid models, such as a Hill function. For an individual item, statistics about the quantity-effect relationship, such as potency, efficacy, and slope, can be computed based on one or more inferred Hill functions for the item. When considering combined effects of two or more items, the combined effects have the same statistics for each individual item plus other parameters that determine the influence of each item on the other item's response. In the case of combined effects, the relationships between relative quantities and their respective combined effect, herein called a response manifold, typically are described by an N-dimensional variable slope sigmoidal manifold, such as a 2-D Hill surface or higher dimensional manifold. In some implementations the representation of the response manifold can be a matrix of values. In some implementations the representation of the manifold can be a set of values for parameters defining a model of an M-dimensional variable slope sigmoidal manifold.

Machine learning techniques can be challenging to apply to predictions of combined effects of items for several reasons, of which some are the following.

When considering combined effects of items, the interaction of a pair of items may be additive, synergistic, antagonistic, neutral, or nonlinear, and even combinations of these depending on the quantities of the items involved in the interaction. It is possible for items to be synergistic in some quantities and antagonistic in other quantities. For example, two compounds may act synergistically in producing a desired bioactivity in one combination of doses, but in other combinations, they may act antagonistically. If a model generates a singular, summary score, or generates a set of covariant scores, to represent synergy of a set of items, then such a score or sets of scores may not accurately predict combined effects for an arbitrary set of quantities of the set of items.

As described above, the training set is selected from a set of researched items for which quantifiable information about certain properties is known. In the case of combined effects, information may be available for a singular effect of an item, such a bioactivity of a single compound, but likely not for a combined effect of that item with another item. As another example, information may be available for a combined effect for a pair of items in some quantities, but not in other quantities. Thus, the “known” information about the combined effect of a pair of items may be incomplete.

In addition, while some researched items have quantifiable information about certain properties which is known, such quantifiable information is generally obtained through laboratory experiments. Such experiments typically are performed in replicates to capture experimental variation. This experimental variation can be substantial. Variation in measurements also may arise through performing different experiments, or performing experiments with different equipment, source material, or personnel. For example, some laboratory environments are more well controlled and well equipped than others to produce repeatable and reliable measurements. As another example, the nature of a property of an item and how it can be measured can introduce uncertainty in the information available about properties of researched items. Such variation in data in a training set can adversely impact the performance of a trained computational model.

A further problem can arise if a predictive model is not consistent with or does not completely reflect the nature of interaction a pair of items may have. In such situations model performance likely will be adversely impacted. Also, it is likely that different computational models would provide significantly different predictions. Additionally, in some cases the computational models may make predictions that are unrealistic, such that the predicted combined effect is outside of a range of effects that items actually have.

Also, when considering the combined effect of two or more items, substantial differences between items being combined can be relevant. Using the example of compounds, data representing researched compounds may include on one hand known bioactivity of singular compounds in different chemical subclasses, and on the other hand combinations of compounds from certain different chemical subclasses but not all combinations of subclasses. For example, there may be training samples for compounds in class A, for compounds in class B, for combinations of compounds which are both in class A, and for combinations of compounds which are both in class B. This information may not be sufficient to predict the behavior of combinations where one compound is in class A, and another is in class B.

To address such problems, a variety of machine learning techniques can be used.

First, a computational model is constructed to output, and allow supervision based on, the set of values that defines an N-dimensional response manifold representing a combined effect. In two-dimensions, this set of values defines a response surface, which can be represented using a matrix of values sampling the response surface or set of parameters of a function that models the response surface. A matrix of known values for the combined effects of researched items can be used as supervisory information during training.

In some implementations, the training set includes researched items for which the respective quantitative information describing the combined effect of items is incomplete. For example, a matrix of values representing the combined effect may be incomplete. In such implementations, given the data that is known and a model of the interaction of the two researched items, the quantitative information can be processed, for example using interpolation, to complete the set of values representing the combined effect.

In some implementations, the training set includes researched items for which the respective quantitative information describing the combined effect of the items includes multiple distinct measurements of the combined effect of the researched items together in a plurality of different combinations of quantities. Such multiple distinct measurements include experimental error. The computational model is constructed to include parameters representing uncertainty associated with the measurements of combined effects, which also are trained, so that the trained computational model outputs a set of values for these parameters associated with any prediction.

To address the potential problem of a machine learning model modeling interactions of the items incompletely, predictions about combined effects can be generated by different computational models and training sets. To compare predictions among different computational models, some statistics related to the predictions can be computed and reported. A form of ensembling can be used to combine the outputs of multiple models, and statistics related to the ensembling operation also can be computed and reported.

In some of the implementations, the combinatorics problem is effectively addressed by integrating disparate datasets with different experimental setups and training simultaneously across multiple dimensions, thereby improving generalization capabilities.

To address the potential problem of unrealistic predictions, in some implementations, prior information, such as data available for researched items, can be used ensure that the computational model outputs predicted effects within a physically likely range. The prior information can be data representing a predetermined likely distribution of the combined effect. The prior information can be data representing upper or lower bounds, or both, for the combined effect. Such prior information can be used during training or during inference.

These machine learning techniques help to generalize the known information about combinations of researched items so as to enable the computer system to make predictions about novel combinations of items. In an example implementations, a single end-to-end differentiable machine learning model is capable of nominating novel combination therapies while simultaneously predicting their underlying synergistic properties and reconstructing the predicted two-dimensional surface of a cellular response at any arbitrary range of doses.

Accordingly, in one aspect a computer system predicts properties of combinations of items. In one aspect a computer system uses multiple machine learning to predict properties of items, including a property that is an effect of the combination of two items. In one aspect a computer system predicts bioactivity as a combined effect of the presence of two compounds together in or on a living thing. In one aspect, a computer system uses a machine learning model both to nominate beneficial drug combinations and to simultaneously reconstruct their 2D dose response surfaces.

In one aspect, such a computer system comprises a processing system configured to train a computational model using data representing researched items. The data representing researched items includes, for each pair of researched items, respective quantitative information describing a combined effect of the pair of researched items together in a plurality of different combinations of quantities. The respective quantitative information for each pair of researched items comprises a respective set of values describing a response manifold. Data representing a plurality of potential candidate items is input to the trained computational model such that the trained computational model outputs a result set. The result set includes respective predicted information, for each predicted candidate item from among the plurality of potential candidate items, describing a combined effect of at least the predicted candidate item and another item together in a plurality of different combinations of quantities. The respective predicted information for each predicted candidate item comprises a respective set of values describing an N-dimensional variable slope sigmoidal manifold, wherein N is a positive integer greater than or equal to two.

In one aspect, such a computer system comprises a processing system configured to instantiate a computational model having inputs receiving at least first data representing a first item, and having an output providing predicted information describing a predicted combined effect of the first item and a second item together in a plurality of different combinations of quantities of the first item and the second item. The predicted information describing the predicted combined effect comprises a set of values describing an N-dimensional variable slope sigmoidal manifold, wherein N is a positive integer greater than or equal to two. The computational model is trained using data representing a plurality of researched items. The data representing the plurality of researched items includes, for each pair of researched items, respective quantitative information describing a combined effect of the pair of researched items together in a plurality of different combinations of quantities wherein the respective quantitative information for each pair of researched items comprises a respective set of values describing a response manifold.

In one aspect, such a computer system comprises a processing system configured to configure a trained computational model having inputs receiving at least first data representing a first item, and having an output providing predicted information describing a predicted combined effect of the first item and a second item together in a plurality of different combinations of quantities. The predicted information describing the predicted combined effect comprises a set of values describing an N-dimensional variable slope sigmoidal manifold, wherein N is a positive integer greater than or equal to two. Data representing a plurality of potential candidate items is input to the inputs of the trained computational model such that the output of the trained computational model provides a result set. The result set includes respective predicted information, for each predicted candidate item from among the plurality of potential candidate items, describing a combined effect of at least the predicted candidate item and another item together in a plurality of different combinations of quantities.

In any of the foregoing, the inputs of the computational model can include inputs to receive second data representing the second item.

In any of the foregoing, the quantitative information comprising the set of values describing the response manifold describing the combined effect can include a respective matrix of values.

In any of the foregoing, the quantitative information comprising the set of values describing the response manifold describing the combined effect can include a respective set of values for a set of parameters of a model describing an M-dimensional variable slope sigmoidal manifold.

In any of the foregoing, the predicted information comprising the set of values describing the response manifold describing the combined effect can include a respective set of values for a set of parameters of a model describing an N-dimensional variable slope sigmoidal manifold.

In any of the foregoing, the predicted information comprising the set of values describing the response manifold describing the combined effect can include a respective matrix of values.

In one aspect, such a computer system comprises a processing system configured to train a computational model using data representing researched items. The data representing researched items includes, for each pair of researched items, respective quantitative information describing multiple distinct measurements a measured combined effect of the pair of researched items together in a plurality of different combinations of quantities, such that the multiple distinct measurements include experimental error. The respective quantitative information for each pair of researched items comprises a respective set of values describing a response manifold describing the combined effect. The computational model includes parameters representing uncertainty associated with measurements of the combined effect. Data representing a plurality of potential candidate items is input to the trained computational model such that the trained computational model outputs a result set. The result set includes respective predicted information, for each predicted candidate item from among the plurality of potential candidate items, describing a combined effect of at least the predicted candidate item and another item together in a plurality of different combinations of quantities. The respective predicted information for each predicted candidate item comprises a respective set of values for the parameters representing uncertainty associated with measurements of the combined effect.

In one aspect, such a computer system comprises a processing system configured to instantiate a computational model having inputs receiving first data representing a first item, and having an output providing predicted information describing a predicted combined effect of the first item and a second item together in a plurality of different combinations of quantities of the first item and the second item. The predicted information describing the predicted combined effect comprises a set of values describing a response manifold describing the predicted combined effect. The computational model includes parameters representing uncertainty associated with measurements of the combined effect. The computational model is trained using data representing a plurality of researched items. The data representing the plurality of researched items includes, for each researched item, respective quantitative information describing a combined effect of the researched item and the second researched item together in a plurality of different combinations of quantities of the researched item and the second item.

In one aspect, such a computer system comprises a processing system configured to configure a trained computational model having inputs receiving first data representing a first item, and having an output providing predicted information describing a predicted combined effect of the first item and a second item together in a plurality of different combinations of quantities. The predicted information describing the predicted combined effect comprises a set of values describing a response manifold describing the combined effect. The trained computational model includes parameters representing uncertainty associated with measurements of the combined effect. Data representing a plurality of potential candidate items is input to the inputs of the trained computational model such that the output of the trained computational model provides a result set. The result set includes respective predicted information, for each predicted candidate item from among the plurality of potential candidate items, describing a combined effect of at least the predicted candidate item and another item together in a plurality of different combinations of quantities. The respective predictive information further includes respective values for parameters representing uncertainty associated with measurements of the combined effect.

In any of the foregoing, the mathematical model of the response manifold can be fitted probabilistically.

In any of the foregoing, the quantitative information comprising the set of values describing the response manifold describing the combined effect can include a respective matrix of values.

In any of the foregoing, the quantitative information comprising the set of values describing the response manifold describing the combined effect can include a respective set of values for a set of parameters of a model describing an M-dimensional variable slope sigmoidal manifold.

In any of the foregoing, the predicted information comprising the set of values describing the response manifold describing the combined effect can include a respective set of values for a set of parameters of a model describing an N-dimensional variable slope sigmoidal manifold.

In any of the foregoing, the predicted information comprising the set of values describing the response manifold describing the combined effect can include a respective matrix of values.

In one aspect, such a computer system comprises a processing system configured to train a computational model using data representing researched items. The data representing researched items includes, for each pair of researched items, respective quantitative information describing a combined effect of the pair of researched items together in a plurality of different combinations of quantities. The supervisory information for training the computational model comprises, for each pair of researched items, a multidimensional representation of the respective combined effect of the pair of researched items together for the plurality of different combinations of quantities. Data representing a plurality of potential candidate items is input to the trained computational model such that the trained computational model outputs a result set. The result set includes respective predicted information, for each predicted candidate item from among the plurality of potential candidate items, describing a combined effect of at least the predicted candidate item and another item together in a plurality of different combinations of quantities.

In one aspect, such a computer system comprises a processing system configured to instantiate a computational model having inputs receiving first data representing a first item, and having an output providing predicted information describing a predicted combined effect of the first item and a second item together in a plurality of different combinations of quantities of the first item and the second item. The predicted information describing the predicted combined effect comprises a multidimensional representation of the predicted combined effect for the plurality of different combinations of quantities of the first item and the second item. The computational model is trained using data representing a plurality of researched items. The data representing the plurality of researched items includes, for each pair of researched items, respective quantitative information describing a combined effect of the pair of researched items together in a plurality of different combinations of quantities. The supervisory information for training the computational model comprises, for each pair of researched items, a multidimensional representation of the respective combined effect of the pair of researched items together for the plurality of different combinations of quantities.

In one aspect, such a computer system comprises a processing system that configures a trained computational model having inputs receiving first data representing a first item, and having an output providing predicted information describing a predicted combined effect of the first item and a second item together in a plurality of different combinations of quantities. Supervisory information used to train the computational model comprises, for each pair of researched items, a multidimensional representation of a respective combined effect of the pair of researched items together for a plurality of different combinations of quantities. Data representing a plurality of potential candidate items is input to the inputs of the trained computational model such that the output of the trained computational model provides a result set. The result set includes respective predicted information, for each predicted candidate item from among the plurality of potential candidate items, describing a combined effect of at least the predicted candidate item and another item together in a plurality of different combinations of quantities.

In any of the foregoing, the multidimensional representation of the respective combined effect of the pair of researched items together for the plurality of different combinations of quantities can include a respective matrix of values.

In any of the foregoing, the multidimensional representation of the respective combined effect of the pair of researched items together for the plurality of different combinations of quantities can include a respective set of values for a set of parameters of a model describing an M-dimensional variable slope sigmoidal manifold, wherein M is a positive integer greater than or equal to two.

In any of the foregoing, the multidimensional representation of the respective combined effect of the pair of researched items together for the plurality of different combinations of quantities can include a respective estimate for the quantitative information describing the combined effect of the pair of researched items together in a plurality of different combinations of quantities based on known data for at least one of the researched items.

In any of the foregoing, the data representing researched items can include quantitative information describing a first effect of the first item in a first plurality of quantities and quantitative information describing a second effect of the second item in a second plurality of quantities. Generating the initial estimate of the quantitative information describing the combined effect can be based on the first effect and the first plurality of quantities for the first item and the second effect and the second plurality of quantities for the second item.

In any of the foregoing, generating the initial estimate can include interpolating the quantitative information to fit a mathematical model describing an M-dimensional variable slope sigmoidal manifold, wherein M is a positive integer greater than or equal to two to obtain a set of values for a set of parameters of the mathematical model.

In one aspect, such a computer system comprises a processing system configured to train a computational model using data representing researched items. The data representing researched items includes, for each pair of researched items, respective quantitative information describing a combined effect of the pair of researched items together in a plurality of different combinations of quantities. Data representing a plurality of potential candidate items is input to the trained computational model such that the trained computational model outputs a result set. The result set includes respective predicted information, for each predicted candidate item from among the plurality of potential candidate items, describing a combined effect of at least the predicted candidate item and another item together in a plurality of different combinations of quantities. The respective predicted information describing the combined effect is maintained within a physically likely range based on data representing a predetermined likely distribution of the combined effect.

In one aspect, such a computer system comprises a processing system configured to train a computational model using data representing researched items. The data representing researched items includes, for each pair of researched items, respective quantitative information describing a combined effect of the pair of researched items together in a plurality of different combinations of quantities. Predicted information output by the computational model during training and describing a combined effect of a pair of researched items is maintained within a physically likely range based on data representing a predetermined likely distribution of the combined effect. Data representing a plurality of potential candidate items is input to the trained computational model such that the trained computational model outputs a result set. The result set includes respective predicted information, for each predicted candidate item from among the plurality of potential candidate items, describing a combined effect of at least the predicted candidate item and another item together in a plurality of different combinations of quantities.

In one aspect, such a computer system comprises a processing system configured to instantiate a computational model having inputs receiving first data representing a first item, and having an output providing predicted information describing a predicted combined effect of the first item and a second item together in a plurality of different combinations of quantities of the first item and the second item. The computational model is trained using data representing a plurality of researched items. The data representing the plurality of researched items includes, for each pair of researched items, respective quantitative information describing a combined effect of the pair of researched items together in a plurality of different combinations of quantities. Predicted information output by the computational model during training and describing a combined effect of a pair of researched items is maintained within a physically likely range based on data representing a predetermined likely distribution of the combined effect.

In one aspect, such a computer system comprises a processing system that configures a trained computational model having inputs receiving first data representing a first item, and having an output providing predicted information describing a predicted combined effect of the first item and a second item together in a plurality of different combinations of quantities. Data representing a plurality of potential candidate items is input to the inputs of the trained computational model such that the output of the trained computational model provides a result set. The result set includes respective predicted information, for each predicted candidate item from among the plurality of potential candidate items, describing a combined effect of at least the predicted candidate item and another item together in a plurality of different combinations of quantities. The respective predicted information describing the combined effect is maintained within a physically likely range based on data representing a predetermined likely distribution of the combined effect.

In any of the foregoing, the data representing a predetermined likely distribution of the combined effect can include an upper bound and a lower bound.

In any of the foregoing, the predicted information describing the combined effect can be maintained within the physically likely range by a applying a thresholding operation to the predicted information output by the model.

In any of the foregoing, the predicted information describing the combined effect can be maintained within the physically likely range by applying a prior distribution within the computational model that outputs the predicted information.

In any of the foregoing, the predicted information describing the combined effect can be maintained within the physically likely range by applying a prior distribution within the computational model that outputs the predicted information.

In any of the foregoing, the predicted information describing the combined effect can be maintained within the physically likely range by adjusting values for parameters of the computational model during training according to a prior distribution.

In any of the foregoing, the data representing researched items can include quantitative information describing a first effect of the first item in a first plurality of quantities and quantitative information describing a second effect of the second item in a second plurality of quantities, and wherein the generating the initial estimate of the quantitative information describing the combined effect can be based on an estimate of a matrix of values based on the quantitative information describing a first effect of the first item in a first plurality of quantities and quantitative information describing a second effect of the second item in a second plurality of quantities.

In any of the foregoing, the M-dimensional variable slope sigmoidal manifold can include a sigmoidal surface.

In any of the foregoing, the N-dimensional variable slope sigmoidal manifold can include a sigmoidal surface.

In any of the foregoing, the quantitative information describing the combined effect can include a set of values for a set of parameters of a mathematical model for a response manifold describing the combined effect, and the computational model can include parameters representing uncertainty associated with measurements of the combined effect.

In any of the foregoing, the predicted information can include parameters representing uncertainty associated with measurements of the combined effect.

In any of the foregoing, the predicted information describing the combined effect can include a set of values for a set of parameters of a model describing an N-dimensional variable slope asymmetric manifold, wherein N is a positive integer greater than or equal to two.

In any of the foregoing, supervisory information for training the computational model can include a multidimensional representation of the combined effect for the plurality of different combinations of quantities of the first item and the second item.

In any of the foregoing, the quantitative information describing the combined effect can include data representing a lower bound for an effect of the first item.

In any of the foregoing, the quantitative information describing the combined effect can include data representing an upper bound for an effect of the first item.

In any of the foregoing, the quantitative information describing the combined effect can include data representing a lower bound for an effect of the second item.

In any of the foregoing, the quantitative information describing the combined effect can include data representing an upper bound for an effect of the second item.

In any of the foregoing, the quantitative information describing the combined effect can include data representing a lower bound for the combined effect.

In any of the foregoing, the quantitative information describing the combined effect can include data representing an upper bound for the combined effect.

In any of the foregoing, the predicted information describing the combined effect can include data representing a lower bound for the combined effect.

In any of the foregoing, the predicted information describing the combined effect can include data representing an upper bound for the combined effect.

In any of the foregoing, the quantitative information describing the combined effect can include data representing a piece-wise continuous function.

In any of the foregoing, the predicted information describing the combined effect can include data representing a piece-wise continuous function.

In any of the foregoing, the data representing researched items can include quantitative information describing a first effect of the first item in a first plurality of quantities and quantitative information describing a second effect of the second item in a second plurality of quantities, and the processing system can be configured to generate the quantitative information describing the combined effect of the first item and the second item together based on the first effect and the first plurality of quantities for the first item and the second effect and the second plurality of quantities for the second item.

In any of the foregoing, generating the quantitative information can include interpolating the quantitative information describing a first effect of the first item in a first plurality of quantities or the quantitative information describing a second effect of the second item in a second plurality of quantities, or both.

In any of the foregoing, generating the quantitative information can include extrapolating the quantitative information describing a first effect of the first item in a first plurality of quantities or the quantitative information describing a second effect of the second item in a second plurality of quantities or both.

In any of the foregoing, the generated quantitative information describing the combined effect can include a set of values for a set of parameters of a mathematical model for a response manifold describing the combined effect, and the computational model can include parameters representing uncertainty associated with measurements of the combined effect.

In any of the foregoing, the M-dimensional variable slope sigmoidal manifold can be defined by a two-dimensional Hill function.

In any of the foregoing, the M-dimensional variable slope sigmoidal manifold can be defined by a combination of a first one-dimensional variable slope sigmoidal curve and a second one-dimensional variable slope sigmoidal curve.

In any of the foregoing, the N-dimensional variable slope sigmoidal manifold can be defined by a two-dimensional Hill function.

In any of the foregoing, the N-dimensional variable slope sigmoidal manifold can be defined by a combination of a first one-dimensional variable slope sigmoidal curve and a second one-dimensional variable slope sigmoidal curve.

In any of the foregoing, the mathematical model can be defined by fitting the model probabilistically, or fitting the model using other methods.

In any of the foregoing, the M-dimensional variable slope sigmoidal manifold can represent a combination of a first dose-response curve for the first item and a second dose-response curve for the second item.

In any of the foregoing, the N-dimensional variable slope sigmoidal manifold can represent a combination of a first dose-response curve for the first item and a second dose-response curve for the second item.

In any of the foregoing aspects, the researched compounds can include one or more of small synthetic molecules or drugs.

In any of the foregoing aspects, the potential candidate compounds can include one or more of proteins found in food, compounds found in food, compounds that a generally recognized as safe for human consumption, or large naturally occurring molecules.

In any of the foregoing aspects, information characterizing bioactivity for a compound in the plurality of research compounds can include measured and quantified bioactivity related to a protein in response to presence of the compound in a living thing.

In any of the foregoing aspects, the selected type of bioactivity can include bioactivity related to a selected protein in response to presence of a compound in a living thing. The selected subset of the plurality of researched compounds includes researched compounds having information characterizing bioactivity related to the selected protein.

In any of the foregoing aspects, bioactivity related to a protein can include bioactivity related to a concentration of the protein present in a living thing.

In any of the foregoing aspects, bioactivity can include bioactivity related to a health condition of a living thing, such as a concentration of protein present in the living thing.

In any of the foregoing aspects, the living thing can include one or more of plants, mammals, animals, or humans.

In any of the foregoing aspects, the information characterizing bioactivity can include a measured concentration of a protein in response to presence of a measured amount of a compound. The information can include an amount in a continuous or semi-continuous range indicating a concentration of an item in a sample. The information can include a concentration of another item related to the amount of protein present in a sample.

In any of the foregoing aspects, a computational model can be designed to predict whether the candidate compounds interact directly or indirectly with, or independently of, the respective selected protein. The interaction can be positive or negative. a computational model can be designed to predict whether the candidate compounds interact, when present with another compound, with the respective selected protein.

In any of the foregoing aspects, querying can include identifying one or more of: compounds that interfere with activity of a drug, foods containing compounds that interfere with activity of a drug, compounds that enhance activity of a drug, foods containing compounds that enhance activity of a drug.

In any of the foregoing aspects, querying can include aggregating interaction information for a plurality of compounds to characterize an overall effect of the plurality of compounds with respect to a health condition, or with respect to a drug.

The following Detailed Description references the accompanying drawings which form a part of this application, and which show, by way of illustration, specific example implementations. Other implementations may be made without departing from the scope of the disclosure.

### BRIEF DESCRIPTION OF THE DRAWINGS

FIG. 1 is a data flow diagram of an example implementation of a computer system that uses machine learning techniques to predict combined effects of items.

FIG. 2 is an example implementation of a data structure for data representing researched items, potential candidate items, and predicted candidate items.

FIG. 3 is an illustration of data representing a combined effect.

FIG. 4 is a data flow diagram of an example implementation of a computer system that incorporates a computational model for predicting a combined effect.

FIG. 5 is a flowchart describing an example operation of a computational model for predicting a combined effect.

FIG. 6 is a data flow diagram of an example implementation of a computer system that incorporates a computational model using interpolated data for researched items.

FIG. 7 is a data flow diagram of an example implementation of a computer system that incorporates a computational model using uncertainty modeling.

FIG. 8 is a flowchart describing an example operation of a computational model using uncertainty modeling.

FIG. 9 is a data flow diagram of an example implementation of a computer system that incorporates a computational model using a prior information.

FIG. 10 is a block diagram of an example general purpose computer.

FIG. 11 is a data flow diagram of an example implementation of a combinatoric machine learning model that predicts parameters representing a response surface.

In the drawings, in the data flow diagrams, a parallelogram indicates an object that is an input to a system that manipulates the object or an output of such a system, whereas a rectangle indicates the system that manipulates that object.

### DETAILED DESCRIPTION

Machine learning techniques can be used to build a computer system that can predict properties of items. To do so, the computer system has access to data representing a set of researched items for which a property is known. The property which a researched item has is one from among a plurality of types of properties. The computer system also has access to data representing potential candidate items. For each potential candidate item, respective information is not known for at least one property among the plurality of types of properties. The computer system applies machine learning techniques to train a computational model using the data representing the researched items and their known properties, for a plurality of types of properties. The computer system applies the trained computational model to the data representing the potential candidate items. In response, the trained computational model outputs one or more predictions about whether the potential candidate items are likely to have a property from among the plurality of types of properties that the computational model is trained to predict.

The “potential candidate items” that are predicted by the computational model to have a property become “predicted candidate items” for that property. Laboratory or other experiments can be performed to verify the predictions output by the trained computational model. Verification transforms a prediction about a predicted candidate item into quantitative information about a known property of a researched item. Thus, note that an item can be a potential candidate item with respect to one type of property that is not known for the item, yet can also be a researched item with respect to another different type of property that is known for the item, and yet can also be a predicted candidate item with respect to yet another different type of property.

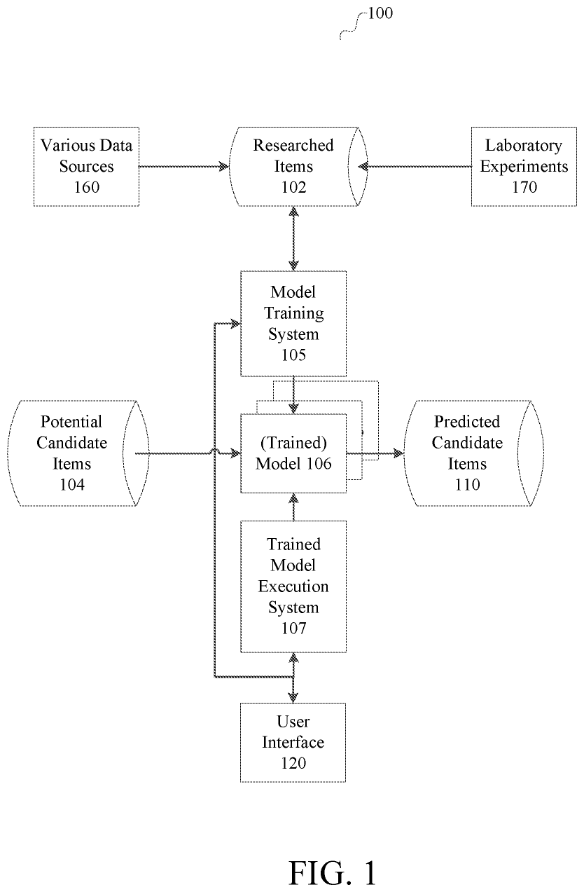

Referring now to the data flow diagram of FIG. 1, an example implementation of a computer system that uses machine learning techniques to predict combined effects of items will now be described.

The computer system 100 has access to data 102 representing a set of researched items. A researched item is an item for which a property is known. The property which a researched item has is one from among a plurality of types of properties. The computer system 100 also has access to data 104 representing potential candidate items. A potential candidate item is an item for which the property is not known. Information about researched compounds can come from a various data sources 160, examples of which are described in more detail below, or from laboratory experiments 170, or both.

The computer system applies machine learning techniques, implemented by a model training system 105, to train a computational model 106 using data 102 representing the researched items and their known properties. The computer system, using the trained model execution system 107, applies the trained computational model 106 to data 104 representing potential candidate items. In response, the trained computational model outputs data 110 representing one or more predictions about whether the potential candidate items are likely to exhibit a property. Those potential candidate items that are predicted to have a property are predicted candidate items for that property.

One or more user interfaces 120 can be provided to enable a user to access the computer system 100. For example, a user interface can be provided to identify researched items for training sets for use by model training system 105. A user interface can be provided to identify potential candidate items to input to a trained model 106 to obtain predictions. A user interface can be provided to search the information about predicted candidate items 100 to identify items to be submitted for laboratory experiments (170).

Laboratory or other experiments can be performed to verify the predictions output by the trained computational model. The computer system 100 can have an input interface (not shown) through which data can be received that includes information characterizing verified properties based on laboratory or other experiments 170 performed to verify predictions. Through this interface, such data can be stored in the database 102 of researched items, thus transforming a prediction into quantitative information about a property of a researched item.

Items can include any of a variety of physical items, which may include machines, articles of manufacture, or compositions of matter, or any combination of these. Such physical items include compounds, and combinations of compounds, including various forms of such combinations (e.g., mixtures, solutions, alloys, conglomerates) or structure of such combinations (e.g., mechanical, electrical, or other interconnection).

As used herein, a compound is any molecular structure. Compounds can be described by their source, such as a living thing, such as a plant or animal, naturally occurring or manufactured, industrial, pollutant, food, and so on. Compounds also can be described by their typical activity with respect to other compounds, such as binding, agonist, antagonist, increasing response, decreasing response, partial agonist, partial antagonist, inverse agonist/antagonist, transcription modulation, phosphorylation, sequestration, catalyst, and so on. Compounds can be described by their compositional type, such as small molecule, macromolecule, large molecule, or polymer. Molecules may be organic or inorganic. Example organic molecules include but are not limited to lipids, alkaloids, nucleic acids, polypeptides, and so on. Example polymers include but are not limited to proteins, peptides, nucleic acids (e.g., RNA, DNA, or fragments thereof), glycan, or any combinations of the above.

Properties of items can include mechanical, optical, electrical, magnetic, electrooptical, electromagnetic, chemical, biological, or other properties (e.g., liquid, gas, solid, or other state) or any combination of these. To be useful in a machine learning context, the property of an item is quantifiable, and combined effects are quantifiable. The property can be any quantifiable information about the item which can be measured. For example, a property of an item can be quantified through laboratory experiments, field measurements, or other techniques. For example, a property of a compound can be a measured concentration of a response, such as a protein, in a sample in response to presence of a measured amount of the researched compound. Information that quantifies a property can be a value in a continuous range, in a piece-wise continuous range, or in a discrete range. The property can be measured with any sensor appropriate to the nature of the property.

Non-limiting examples of properties of a compound include, but are not limited to physical properties, reactivity, bioactivity (i.e., a quantifiable biological response of a living thing when the compound is present in or on the living thing), or biological properties. Example physical properties include molecular weight, protonation state, salt state, melting point, crystal structure, boiling point, density, length, volume, pH, and so on. Examples of reactivity include side chains (e.g., OH, COOH, NH₂, etc.), a number of bonds, a number of rotatable bonds, and so on. Examples of biological properties include the source of the compound (e.g., plant, animal, fungus, etc.), metabolism, and so on. A biological property can characterize a biological response as positive (i.e., healthy), negative (i.e., unhealthy), or neutral, or a combination of responses, such as a positive response such as reduction of a symptom and a simultaneous negative response such as a side effect. The information that quantifies a property can be a measurement of a characteristic of a reaction. This information can represent, for example, a concentration of a protein, a concentration of another item related to an amount of a protein, a concentration of RNA expression data, a readout from a sensor, such as luminescence, fluorescence, or radiation, or any other characteristic of the reaction that can be measured.

Data representing an item typically includes, or is processed to provide, a set of values for a set of features which distinguish the item from other items. Such features are used in machine learning as inputs to train a computational model or to use a trained computational model. The features may be a part of the data representing the item or may be derived from data representing the item.

For example, for a compound, the data representing a compound typically includes data defining the molecular structure of the compound. Data defining molecular structure of a compound can include any one or more of data representing: a molecular formula for the compound, a name for the compound, any isomers of the compound, a two-dimensional chemical structure of the compound, a SMILE string, three dimensional conformations of the molecule of the compound, any chemical property descriptors such as an RDKit descriptor, molecular properties, such as crystal structure, molecular weight, solubility. Such data, or any data resulting from transformation of such information, can be used as features which can be input to a machine learning model. As an example, features representing a compound can include a mapping onto a protein-protein interaction graph based on known compound-protein interactions, which is an ‘impact’-based featurization. As another example, features derived from functional RNA data for a compound can include an inherent composition based on primary sequence and secondary structure such as the presence of certain key motifs and kmers, or a transcriptomic differential expression profile when the functional RNA is perturbing a basal cell line.

In some practical applications, combinations of items and effects produced by such combinations of items in different quantities are considered. In such applications, the property of an item is a combined effect of the item with another item in two or more distinct combinations of different quantities. A combined effect, over a range of possible quantities of the first item and a range of possible quantities of the second item, may be unknown, partially known, or substantially known.

In the case of combined effects, interaction of a pair of items may be additive, synergistic, antagonistic, neutral, or nonlinear, and even combinations of these depending on the quantities of the items involved in the interaction. In particular, it is possible for items to be synergistic in some quantities and antagonistic in other quantities. For example, two compounds may act synergistically in producing a desired bioactivity in one combination of doses, but in other combinations, they may act antagonistically.

Generally, a combined effect can be represented as a response manifold over a domain of two or more inputs. With two inputs, the combined effect can be represented as a response surface. Data representing the combined effect can be a matrix of values representing a mapping of different quantities of different items to the respective combined effects of those items in those different quantities. In some implementations, such a matrix may be incomplete based on known information about researched items. For example, information may be available for a combined effect for a pair of items in some quantities, but not in other quantities. In some implementations, the data representing the combined effect can be a set of values for a set of parameters of a model that describes the response manifold.

In such applications, a property of a researched item which is known can be a combined effect of a first item and a second item together. In such applications, a property of a predicted candidate item can be a predicted combined effect of at least a first item and a second item. In some implementations, a computer system may represent the predicted candidate item as a first item, and its property is its combined effect with the second item. In some implementations, a computer system may represent the predicted candidate item as the combination of the first item and the second item, and its property is their combined effect. Other implementations are possible. It is possible that this combined effect may be known for a plurality of different combinations of quantities of the first item and second item together. For example, the combined effect may be known for the first item in a first quantity together with a quantity of the second item and the combined effect may be known for the first item in a second quantity together with the quantity of the second item. For example, the combined effect also may be known for the first item in the first quantity together with a different quantity of the second item and the combined effect may be known for the first item in the second quantity with the different quantity of the second item.

Items are “together” when they jointly or simultaneously affect a property of an item. For example, two components in an alloy jointly affect the property of the alloy. As another example, two sets of computer program instructions executed in a computer jointly affect the power consumption and/or performance of the computer. As another example, in the context of compounds and biological response, an example of when compounds are “together” is when a concentration of molecules of the first compound and a concentration of molecules of the second compound are temporally coincident, or simultaneously present, at a same cell or collection of cells in or on the living thing, or in an in vitro or ex vivo sample of such cells, or in representative cells in vitro. Thus, doses may not be simultaneously delivered for compounds to be together, because compounds may reside in or on the living thing for some time until they are absorbed, excreted, broken down, or otherwise are no longer present. Thus, a concentration of a compound can be represented as a function of time in response to a dose, or as a dose amount, or other suitable representation, which can depend on the nature of a test to be performed to measure bioactivity, e.g., in vivo vs. in vitro testing. In a biological system, a compound may be present in a time-varying concentration due to biochemical processes.

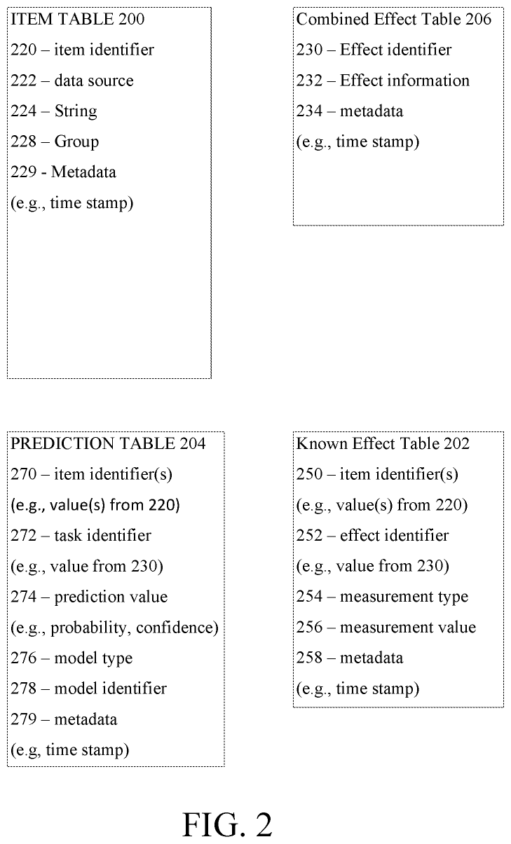

Having now described items, properties of items, and combined effects, and the relationship among researched items, potential candidate items, and predicted candidate items, and an architecture of a computer system in FIG. 1, illustrative data structures for an example implementation of such a computer system for the purposes of researching combined effects of items will now be described in connection with FIG. 2. Such data structures can be implemented, for example, using one or more tables in a relational database, or using one or more data objects in an object-oriented database, or using one or more documents in a NoSQL database, or by using data structures allocated in memory for an executing computer program, or by using any data structures implemented through other programming techniques. The use of database tables in the following examples is merely illustrative.

As shown in FIG. 2, an item table 200 can be used to represent all items, whether researched items, potential candidate items, or predicted candidate items. Another table, herein called a known effect table 202, includes data representing information characterizing properties, such as combined effects of items. Thus, if an item has known property, making it a researched item with respect to that property, then the item has an entry in the known effect table 202 which includes information characterizing that property. Another table, herein called a prediction table 204, includes data representing predicted properties for items. Thus, if an item has been predicted to have a property, making it a predicted candidate item with respect to that property, then the item has an entry in this table that includes information, herein called prediction data, describing that prediction.

In the example shown in FIG. 2, one kind of property that can be represented in the known effect table 202 and the prediction table 204 is a combined effect. The property of interest is the combined effect of an item together with another item. In FIG. 2, a combined effect table 206 includes data representing types of combined effects. Yet other tables may be referenced to store information about other kinds of properties of items.

In the example of a combined effect, the known effect table 202 includes entries, with each entry associating an item with another item, and the quantifiable data describing the known combined effect of those two items together. Similarly, in the example of a combined effect, the prediction table 204 includes entries, with each entry associating an item with another item with which it is predicted to have a combined effect, and data about that prediction.

Additional details of an example implementation of the tables in FIG. 2 will now be described. It should be understood that this example is merely illustrative, as a variety of information can be stored in the database in diverse ways.

In this example, item table 200 includes data representing each item. For each item, information such as an identifier 220, can be stored. This identifier can be used as a primary join key with other tables. A suitable identifier is any form of data that can uniquely identify an item and distinguish the item from among other items in the system. One or more of such identifiers can be stored, allowing processing of the table in diverse ways.

The data representing an item can include an indicator 222 of a source, such as a database or laboratory experiment, from which information about the item has been obtained. The data representing the item can include, for example, a string 224 of characters providing a name of or a description of the item. One or more of such strings can be stored. The string can directly or indirectly provide information about the item. For example, the string may be a reference to a data file defining information about the item.

The data representing an item can include group information 228. Items can be placed into groups, such as by categories, classes, or ontologies. A plurality of distinct groups can be defined. An item can be placed into one or more groups. Grouping of items enables other advantageous operations to be performed in the context of training and using computational models. For example, when specifying train/validate/test splits, placing members of the same group into the same split may reduce overestimating generalization, because predictions for members of the same group may be expected to be similar.

Other metadata 229 about the data representing the item can be stored. For example, a time stamp can be stored indicating when the entry for the item was created, the last time the data representing the item was modified or accessed. A variety of other metadata can be stored. For example, metadata about provenance of data stored in the system can be included, in addition to its source 222.

In the example shown in FIG. 2, a combined effect table 206 includes data representing combined effects. For each combined effect, the data representing the combined effect includes an identifier 230. This identifier 230 can be used as a primary join key with other tables. Any other suitable identifier that uniquely identifies the combined effect can be used. For each effect, the data representing the effect can include data 232 describing the combined effect. Any other information about the combined effect can be stored in the database. Other metadata 234 about the data representing the combined effect can be stored. For example, a time stamp can be stored indicating when the entry for the effect was created, the last time the data representing the effect was modified or accessed.

In the example shown in FIG. 2, known effect table 202 includes data representing known combined effects of items. In some implementations, this table can associate identifiers of items with identifiers related to combined effects with which those items are related.

In known effect table 202, an item identifier field 250 stores one or more identifier(s), e.g., item identifier 220, of one or more item(s). The effect identifier field 252 stores an identifier related to the combined effect. For example, this field can store an item identifier 220 of a second item for which the first item 250 is known to have a combined effect, or an effect identifier 230.

The known effect table 202 further associates these identifiers with information characterizing the combined effect. For example, data indicating a type 254 of measurement, assay, or experiment used, and any value 256 resulting from that measurement, assay, or experiment can be stored. Other metadata 258 about the known effect can be stored. For example, time stamps can be stored indicating when the data was created or the last time this data was modified or accessed.

In the example shown in FIG. 2, prediction table 204 includes data representing predicted combined effects of items. In some implementations, this table can associate identifiers of items with identifiers of combined effects predicted for those items. Data in the prediction table 244 are populated as a result of training a computational model using data about selected researched items and a selected type of property, and then applying the trained model to data about selected potential candidate items.

In prediction table 204, an item identifier field 270 stores one or more identifiers, e.g., 220, of the items. The task identifier field 272 stores an identifier related to the predicted combined effect. For example, this field can store an item identifier 220 of a second item for which the first item 270 is predicted to have a combined effect, or an effect identifier 230.

Prediction table 204 further associates these identifiers with information characterizing the predicted combined effect. For example, this information can include a prediction value 274 representing the combined effect, and a class 276 indicating a type of machine learning model that generated the prediction, to help interpret the prediction value. Different types of machine learning models generate different kinds of prediction values 274, such as a probability, a confidence, a classification, or other output or combination of outputs. In some implementations, the prediction value can be the set of values for a set of parameters describing a Hill model representing the response manifold for different quantities of the items and their result combined effects, such as a dose-response curve. An identifier 278 of the computational model that resulted in this prediction also can be stored Other metadata 279 about the prediction can be stored. For example, a time stamp can be stored indicating when the data was created or the last time this data was modified or accessed.

For combined effects, consider on the one hand the data representing researched items in the known effects table 202, and on the other hand, data representing predictions in the prediction table 204.

In a prediction table 204, the item identifier 270 can include an identifier that represents a specific combination of items, e.g., the pair of identifiers (i.e., values from field 220) for the two (or more) items. Alternatively, a combination table (not shown) can represent combinations of items, with an identifier for a combination that is mapped to two (or more) items by the respective identifiers for those items. The prediction value 274 for a combination can include a representation of their predicted combined effect, such as the set of values for a set of parameters describing a model representing this combined effect.

Similarly, data about the known combined effects of items is found in the known effect table 202. In some examples described herein, the known effect (e.g., a measurement value 256 for an item 250) may be the effect for a singular item. In some cases, the known effect is for a combination of items together. This combined effect can be represented in the known effect table 202 by having the item identifier 250 can include an identifier that represents a specific combination of items, e.g., the pair of identifiers (i.e., values from field 220) for the two (or more) items. Alternatively, a combination table (not shown) can represent combinations of items, with an identifier for a combination that is mapped to two (or more) items by the respective identifiers for those items. The measurement value 256 for a combination of items can include a representation of their combined effect. In some implementations, the data representing a combined effect can be the set of values for a set of parameters describing a model representing this combined effect. In some implementations, a matrix of measurements can be used. For example, the data about a combined effect can be represented as a matrix of values representing a mapping of different quantities of different items to the combined effect of those items in those different quantities. In some implementations, such a matrix may be incomplete.

The training and use of a computational model which accesses and uses the data in the item table 200, known effect table 202, and combined effect table 206, to generate the data in prediction table 204, will now be described in more detail by way of an example, illustrative implementation.

As noted above, using a computational model and a training set of data, e.g., data about researched items, a model training system trains the computational model using a machine learning algorithm, features derived from the training set, and supervisory information available for or derived from the training set. The trained computational model is then applied to a target data set, e.g., a set of potential candidate compounds, to make predictions about the target data set.

A computational model used in a machine learning application typically computes a function of a set of input features, which may be a linear or non-linear function, to produce an output. The function typically is defined by mathematical operations applied to a combination of a set of parameters and the set of input features. Machine learning involves adjusting the set of parameters to minimize errors between the function as applied to a set of input features for a set of training samples and known outputs (supervisory information) for that set of training samples. The output of the computational model typically is a form of classification or prediction.

Such computational models are known by a variety of names, including, but not limited to, classifiers, decision trees, random forests, classification and regression trees, clustering algorithms, predictive models, neural networks, genetic algorithms, deep learning algorithms, convolutional neural networks, artificial intelligence systems, machine learning algorithms, Bayesian models, expert rules, support vector machines, conditional random fields, logistic regression, maximum entropy, among others.

The output of a computational model is a prediction value indicative of whether, or to what extent, an item has, if any, a selected property. This prediction can be in the form of, for example, a probability between zero and one, or a binary output, or a score (which may be compared to one or more thresholds), or other format. As a specific example, for a single item and corresponding property, values for parameters describing a one-dimensional Hill model, or other relevant model, may be known or predicted. For combined effects, values for parameters describing a two-dimensional Hill model, or other relevant model, may be known or predicted. The output can be accompanied by additional information indicating, for example, a level of confidence in the prediction. The output typically depends on the kind of computational model used.

A training set generally comprises a set of samples for which respective information about each sample is known, i.e., a set of researched items. Data called “features” are derived from information available about the samples the training set. These features are used as inputs to a computational model. The known information for the samples, typically called “labels,” i.e., the information characterizing the known properties of the researched items, provides the supervisory information for training. The supervisory information typically corresponds to the desired outputs of a computational model. A computational model has parameters that are adjusted by the training algorithm so that the outputs of the computational model, in response to the features for the samples in the training set, correspond to the supervisory information for those samples. Most training algorithms divide the training set into training data and validation data. Given a trained computational model, the trained computational model can be applied to features derived from the data for potential candidate items. The trained computational model provides an output indicative of a prediction about the potential candidate item.

Using the example data structures shown in FIG. 2, data for a training set can be specified by a query (or an identifier for such a query) on the item table 200 joined with entries from the known effect table 202 that contain one or more selected values as the effect identifier. Similarly, data for the potential candidate items can be specified by a query (or an identifier for such a query) on the item table 200 for items which are not in the training set, and which satisfy any other criteria desired. In the case of combined effects, it is possible to specify items for which the desired property is known for that item singularly, but not in combination with other items. The computational model can treat the combined effect of a combination of such items as being represented by an incomplete matrix.

Given a training set, the model training system 105 (FIG. 1) can train a computational model Generally, a training algorithm applies, as inputs to the computational model, features derived from the data representing the researched items in the training set. Outputs from the computational model are obtained and compared to the supervisory information corresponding to those inputs. Parameters of the computational model are modified so as to reduce the errors between the outputs obtained and the supervisory information. The training algorithm involves iterating these steps of applying, comparing, and modifying until such errors are sufficiently reduced.

After the computation model is trained, the trained model execution system 107 applies the trained computational model 106 to the data representing some potential candidate items. The trained computational model thus generates and stores a result set. The result set includes a set of predicted candidate items (110 in FIG. 1) identified from among the plurality of potential candidate items as likely to have a property. Such information can be stored, for example, in a data structure such as shown as a prediction table 204 in FIG. 2.

Machine learning techniques can be challenging to apply to predictions of combined effects of items for several reasons, of which some are the following.

When considering combined effects of items, the interaction of a pair of items may be additive, synergistic, antagonistic, neutral, or nonlinear, and even combinations of these depending on the quantities of the items involved in the interaction. It is possible for items to be synergistic in some quantities and antagonistic in other quantities. For example, two compounds may act synergistically in producing a desired bioactivity in one combination of doses, but in other combinations, they may act antagonistically. If a model generates a singular, summary score, or generates a set of covariant scores, to represent synergy of a set of items, then such a score or sets of scores may not accurately predict combined effects for an arbitrary set of quantities of the set of items.

Also, as described above, the training set is selected from a set of researched items for which quantifiable information about certain properties is known. In the case of combined effects, information may be available for a singular effect of an item, such a bioactivity of a single compound, but likely not for a combined effect of that item with another item. As another example, information may be available for a combined effect for a pair of items in some quantities, but not in other quantities. Thus, the “known” information about the combined effect of a pair of items may be incomplete.

In addition, while some researched items have quantifiable information about certain properties which is known, such quantifiable information is generally obtained through laboratory experiments. Such experiments typically are performed in replicates to capture experimental variation. This experimental variation can be substantial. Variation in measurements also may arise through performing different experiments, or performing experiments with different equipment, source material, or personnel. For example, some laboratory environments are more well controlled and well equipped than others to produce repeatable and reliable measurements. As another example, the nature of a property of an item and how it can be measured can introduce uncertainty in the information available about properties of researched items. Such variation in data in a training set can adversely impact the performance of a trained computational model.

A further problem can arise if a predictive model is not consistent with or does not completely reflect the nature of interaction a pair of items may have. In such situations model performance likely will be adversely impacted.

Also, when considering the combined effect of two or more items, substantial differences between items being combined can be relevant. Using the example of compounds, data representing researched compounds may include on one hand known bioactivity of singular compounds in different chemical subclasses, and on the other hand combinations of compounds from certain different chemical subclasses but not all combinations of subclasses. For example, there may be training samples for compounds in class A, for compounds in class B, for combinations of compounds which are both in class A, and for combinations of compounds which are both in class B. This information may not be sufficient to predict the behavior of combinations where one compound is in class A, and another is in class B.

To address such problems, a variety of techniques can be used.

First, a computational model is constructed to output, and allow supervision based on, the set of values that defines an N-dimensional response manifold representing a combined effect. In two-dimensions, this set of values defines a response surface, which can be represented using a matrix of values sampling the response surface, or set of parameters of a function that models the response surface. A matrix of known values for the combined effects of researched items can be used as supervisory information during training.

In some implementations, the training set includes researched items for which the respective quantitative information describing the combined effect of items is incomplete. For example, a matrix of values representing the combined effect may be incomplete. In such implementations, given the data that is known and a model of the interaction of the two researched items, the quantitative information can be processed, for example using interpolation, to complete the set of values representing the combined effect.

In some implementations, the training set includes researched items for which the respective quantitative information describing the combined effect of the items includes multiple distinct measurements of the combined effect of the researched items together in a plurality of different combinations of quantities. Such multiple distinct measurements include experimental error. The computational model is constructed to include parameters representing uncertainty associated with the measurements of combined effects, which also are trained, so that the trained computational model outputs a set of values for these parameters associated with any prediction.

To address the potential problem of a machine learning model modeling interactions of the items incompletely, predictions about combined effects can be generated by different computational models and training sets. To compare predictions among different computational models, some statistics related to the predictions can be computed and reported. A form of ensembling can be used to combine the outputs of multiple models, and statistics related to the ensembling operation also can be computed and reported.

To address the potential problem of unrealistic predictions, in some implementations, prior information, such as data available for researched items, can be used ensure that the computational model outputs predicted effects within a physically likely range. The prior information can be data representing a predetermined likely distribution of the combined effect. The prior information can be data representing upper or lower bounds, or both, for the combined effect. Such prior information can be used during training or during inference.

These machine learning techniques help to generalize the known information about combinations of researched items so as to enable the computer system to make predictions about novel combinations of items.

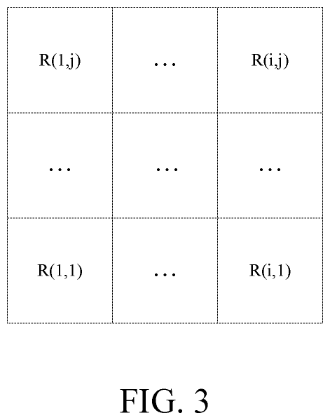

Turning now to FIG. 3, an example implementation of a computational model incorporating modeling of combined effects will now be described.

FIG. 3 is an illustration of a matrix of data representing a combined effect in two dimensions. In this example, quantities of a first item are shown on one axis i, and quantities of a second item are shown on another axis j. The combined effect is represented by a matrix R, with indices i and j, with each value R_{i,j} being indicative of the combined effect of quantity of the first item and a quantity of the second item together on a property of an item.

A computational model is trained using a training set including data representing known combined effects for pairs of items and other data representing those items. Input features include data representing at least one pair of the items, and an output includes one or more values indicative of the combined effect of the pair of items. In training, this output can be compared to supervisory data representing the known combined effects for the pair of items. The result of this comparison can be used to update parameters of the computational model.

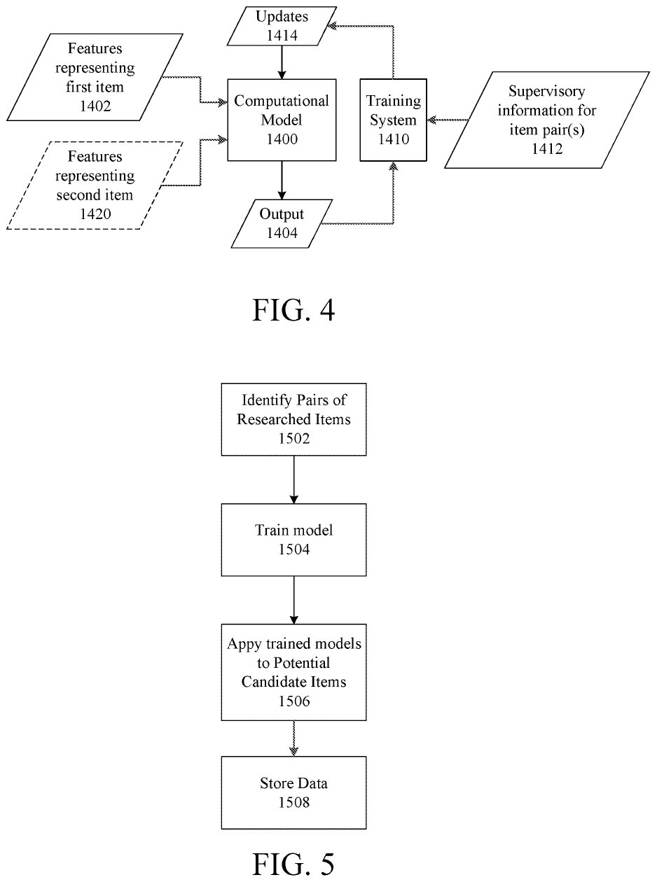

FIG. 4 is a data flow diagram of an example implementation of a computer system that incorporates a computational model for predicting a combined effect.

In this example, a single computational model 1400 is illustrated, but any number of two through any positive integer N models can be used, and ensembled together. In this example, the computational model 1400 receives a set 1402 of input features for a first item (whether a researched item during training, or a potential candidate item during application of the trained model). In some implementations, the computational model has an input to receive a set 1420 of features for a second item (whether a researched item during training, or a potential candidate item during application of the trained model), in the event that multiple second items are being modeled in the computational model 1400. The computational model 1400 provides an output 1404 from which a representation of the combined effect of two items can be derived.

A training system 1410 trains the computational model 1400 using a training set including data representing known combined effects for pairs of items and other data representing those items. Input features include data representing at least one of the items. In some implementations, a single second item can be presumed. In some implementations, if multiple second items are possible, then data 1420 for a second item also is input. The output 1404 includes one or more values from which a representation of the combined effect can be derived.

In training, the output 1404 is compared to supervisory information representing the known combined effects for the known input pair of items. The supervisory information is a matrix representation of the combined effect. The result of this comparison can be used to compute an update 1414 to parameters of the computational model 1400, as described in more detail below.

FIG. 5 is a flowchart describing an example operation for training and using a computational model to predict a combined effect. Training starts with specifying the training set to be used to train a computational model, specifics of the computational model, and a set of potential candidate compounds to which the trained computational model will be applied. At 1502, data representing the researched items are accessed. Then, at 1504, the training system trains the computational model using the training set using the data representing the researched items and their known combined effects. At 1506, a trained model is applied to a set of potential candidate items (PCI). The trained model outputs prediction information for each potential candidate item. The prediction information can be stored (1508) in the database (e.g., data structure 214 in FIG. 2).

In such a system, the computational model can be any model that takes, as an input, a set of features representing a pair of items, and provides as an output, the matrix of values. For training, parameters of the computational model can be updated based on errors between the output matrix of values and the supervisory information, which also is a matrix of values. Two example models of such a type are described in more detail below in connection with an example relating to predicting bioactivity in response to a combination of compounds.

Given a set of items, the data representing those items are processed into a set of values for a set of features. The features used for any particular type of item are selected to capture similarity or differences or both among items of that type. Given the features, a computational model is constructed which predicts the response surface (or response manifold) based on different quantities of the items together. The computation model represents the nature of the interaction between the items in producing the combined effect being predicted. In many biological and chemical processes, this interaction is represented by a 2D Hill surface. Parameters of a model that define the response surface are predicted by the computational model. These parameters can be processed into a matrix of values to allow for supervision of training using a matrix of values.

In the following two examples, which relate to combinations of compounds and bioactivity that occurs in response to the compounds together, two examples of different features are used.

In the first example, data representing compounds is processed into features based on molecular structure information. A technique such as described in Yang, Kevin, et al., “Analyzing Learned Molecular Representations for Property Prediction”, in J. Chem. Inf. Model., 2019, 59, 3370-3388 can be used for this mapping of compound chemical structures into features. A technique such as described in Preuer, Kristina, et al., “DeepSynergy: predicting anti-cancer drug synergy with Deep Learning”, in Bioinformatics, Vol. 34, No. 9, pages 1538-1546 (2018) can be used for this mapping of compound information into features, which is particularly useful in a deep learning machine learning model for predicting combined effects.

In the second example, data representing compounds is processed into features based on a protein-protein interaction framework where compounds are mapped onto a protein network based on known or predicted compound-protein interactions. A graph neural network is trained with a training set to produce a set of features. At inference/prediction time, a set of potential candidate items is input to the graph neural network that generates a set of values for a set of input features for the items. A technique such as described in Cheng, Feixiong, et al., “Network-based prediction of drug combinations”, in Nature Communications, 2019, 10, 1197 can be used for this mapping of compound information into features. A technique such as described in Gonzalez, Guadalupe, et al., “Predicting anticancer hyperfoods with graph convolutional networks”, in Human Genomics, 2021, 15:33 (“Gonzalez 2021”) can be used for this kind of mapping of compound information into features.

More specifically, as an example implementation, an experimentally validated human protein-protein interaction network can be extracted from the STRING database (Szklarczyk, D., et al., “The STRING database in 2023: protein-protein association networks and functional enrichment analyses for any sequenced genome of interest,” in Nucleic Acids Research, 51 (D1):D638-D646, 11 2022. ISSN 0305-1048) as described by Gonzalez 2021. The protein targets of each compound can be extracted from the STITCH database (Szklarczyk, D., et al., “Stitch 5: augmenting protein-chemical interaction networks with tissue and affinity data,” in Nucleic Acids Research, 44 (D1):D380 D384, 2016), CTDbase (Davis, A. P., et al., “Comparative toxicogenomics database (ctd): update 2021”, in Nucleic Acids Research, 49 (D1):D1138-D1143, 2021.) and CHEMBL (Gaulton, A., et al., “The CHEMBL database in 2017”, in Nucleic Acids Research, 45 (D1):D945-D954, 2017).

In such an implementation, each compound can be represented by a graph which encodes the protein-protein interaction network. Each node on the graph denotes a protein on the protein-protein interaction network and each edge denotes an interaction between the two connecting proteins. The binary feature of the node indicates whether the corresponding protein is targeted by the compound and as such the protein targets of the compound can be mapped to the protein-protein interaction network. RNAseq basal expression data can be compiled and processed from the Cancer Cell Line Encyclopedia data set (Ghandi et al., 2019). For example, a set on the order of about 1000 cell lines can be used. In one implementation, a set of the 3,984 most diverse genes were selected as the cell line featurization set. If the exact cell line used in a given dose response assay is not available in the DepMap dataset, the next closest cell line according to cell type can be selected. The representation for each of the compounds can be learned through a shared graph convolution neural network. The learned compound representations are then concatenated together with the cell line representation to form a representation for the drug-cell line combination.

In either case, or with other featurizations, the features are input into a computational model that represents the combined interaction of compounds and that outputs a set of values for a set of parameters which represent the response manifold. In many applications, the response manifold is an N-dimensional variable slope sigmoidal manifold, wherein N is a positive integer greater than or equal to two. When Nis two, such that the combined interaction defines a surface, such a surface is represented by a 2-D Hill surface, which can be specified by a set of parameters.

An example implementation of a set of parameters thar represent a response manifold is based on a mass-action state-transition model of drug combination synergy. An example of such a representation is described in one or more of: Meyer, Christian, et al., “Quantifying Drug Combination Synergy along Potency and Efficacy Axes”, in Cell Syst., 2019 Feb. 27; 8(2): 97-108; and Wooten, David, et al., “MuSyC is a consensus framework that unifies multi-drug synergy metrics for combinatorial drug discovery”, in Nature Communications, 2021, 12:4607 (Wooten 2021). The framework described in these references is herein referred to as the “MuSyC model” and the parameters of this model are referred to as the “MuSyC parameters”. The MuSyC parameters that describe a response surface are the following in Table I:

TABLE I

| | |
|---|---|
| C₁, C₂ | The concentration of drug required to achieve 50% of the maximal effect (i.e., EC₅₀). |
| h₁, h₂ | Hill coefficients for dose-response curves of drug 1 and 2 in isolation. |
| E₀ | The basal effect E_{d} (d1 = d2 = 0). |
| E₁, E₂ | Maximal efficacy of drugs 1 and 2 in isolation. |
| E₃ | Maximal efficacy of the combination of drugs 1 and 2. |
| α₁₂ | Fold change in the potency (C₂) of [d₂] induced by drug 1. |
| α₂₁ | Fold change in the potency (C₁) of [d₁] induced by drug 2. |
| γ₁₂ | Fold change in the cooperativity (h₂) of [d₂] induced by drug 1. |
| γ₂₁ | Fold change in the cooperativity (h₁) of [d₁] induced by drug 2. |

In one implementation, a computational model representing synergy of items is built to output a set of values for these twelve MuSyC parameters based on the features representing items. Given the set of values for the set of parameters predicted by the computational model, this set of values can be converted into a matrix of values representing the combined effect for a pair of items.

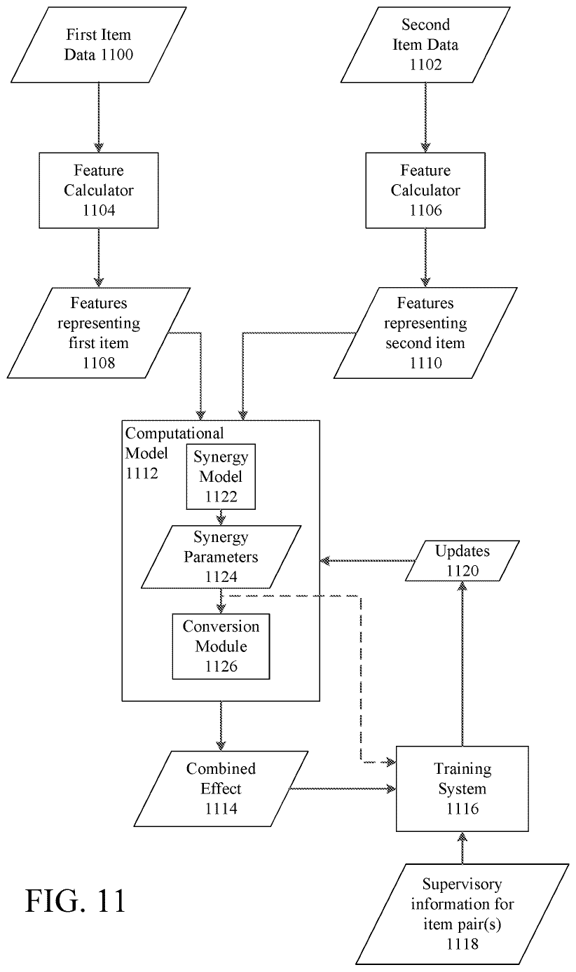

For example, a deep learning predictive machine learning model can be used, and can be configured so that the predictive output of the model takes the form of a predicted response surface defined by MuSyC parameters. For example, a graph neural network (GNN) can be used. The model is constrained to predict 2D Hill surfaces with regularized shape parameter values (i.e., MuSyC parameter values). In the example shown in FIG. 11, these parameters are then processed to generate a matrix of values. These parameters thus serve as an intermediate parameterization, establishing a latent parameterized space of the machine learning model. This machine learning model can be supervised using the parameter values (i.e., supervised in the latent space) or supervised directly on the output matrix of values (i.e., supervised in the output matrix space).

FIG. 11 illustrates this combination of transformations of the data representing items to the data representing the combined effect. Data representing a first item 1100 is processed by a feature calculator 1104 to generate a set of values 1108 for a set of features representing the first item. Similarly, data representing a second item 1102 is processed by a feature calculator 1106 to generate a set of values 1110 for a set of features representing the second item. These features are the inputs to the computational model 1112.

The computational model 1112 includes a synergy model 1122 which, in response to the features representing the first and second items, outputs a set of values for the parameters 1124 representing a response surface, such as the MuSyC parameters, representing the combined effect of those items. Note that the synergy model itself includes parameters (not shown) which affect how the synergy model generates the output values for the parameters 1124. The set of values for the parameters output by the synergy model are inputs to a conversion module 1126. The conversion module computes a matrix of values representing the combined effect, for distinct combinations of different quantities of the two items, based on a function of the parameters 1124.

Instead of predicting the combined effect of the combined items at certain quantities, this computational model predicts parameters that describe the full response surface. Predicting the full response surface provides a better understanding of the combined effects of a range of possible quantities. In the context of drug response for combinations of drugs, this allows a therapeutic window to be more easily identified, and to better understand drug interaction effects. These parameters can be converted into specific values at specific points in a matrix of distinct combinations of quantities of the items.

During training, the output 1114 of the computational model 1112 is provided to a training system 1116, which also receives the supervisory information 1118 for each item pair in a training set. In some implementations, the set of values for the synergy parameters output by the synergy model 1122 can be used as an input to training. Based on the error between the predicted combined effect 1114 and the supervisory information 1118, updates 1120 to the parameters of the computational model are generated and applied to the computational model.

With the two stages of modeling, the system first infers biophysical model parameters, then uses those parameters to generate observables, i.e., a matrix of quantified combined effects at distinct combinations of different quantities. Predicting observables from a parametric model (MuSyC) imposes a strict biological prior on the predictions (regularization of functional form) while also resolving degeneracy in the inference of the latent model parameters. That is, the MuSyC parameters are poorly identified by ground truth observational data, which means that for a given MuSyC parameter, there are many values that can lead to approximately the same surface function. Thus, there are benefits to supervising with a matrix of quantified combined effects as the ground truth data, which is definitely measurable, and not with inferred MuSyC parameters. In particular, the resulting model can infer a range of possible latent MuSyC parameter values corresponding to the quantified observational data.

In some implementations, in training, the MuSyC models can be implemented as a component of the loss function of a deep learning predictive machine learning model. In this implementation, the same parametric model function for the 2D Hill surface can be used, and the prior distributions on the parameter values can also be implemented as penalty terms in the loss function.

In some implementations, a one-stage model is used, where both the output of the model and the supervision of the model use the sets of values for the synergy model parameters. In this case, the known data for a pair of items is processed to generate MuSyC parameters for that pair to use as supervisory information for that pair. In other words, the known data for a pair of items establishes statistically inferred “ground truth” parameter values for tested combinations that can be used as supervising labels during predictive model training.

Some example implementations for fitting known data about combined effects, even if incomplete, to parameters of a synergy model, such as MuSyc parameters, include, but are not limited to: 1. Wooten 2021, the technique described in Meyer, Christian, et al., “Quantifying Drug Combination Synergy along Potency and Efficacy Axes” in Cell Systems, volume 8, issue 2, pages 97-108.e16, February 2019, (fits data to MuSyC parameters), and 2. Hennessey, Violeta, et al. “A Bayesian Approach to Dose-Response Assessment and Synergy and Its Application to In Vitro Dose-Response Studies”, in Biometrics, volume 66, number 4, pages 1275-1283 December 2010 (fits data to parameters of another kind of synergy model).

In an example implementation of this model using the protein-protein interaction information as features, and MuSyC parameters as labels, outputs from the compound graph convolution layers and cell line features are passed separately into two fully connected neural networks to obtain the inferred combination and cell line representations. These representations are then passed through a three-layer fully connected neural network to predict the MuSyC parameters.

In another example implementation of this model using the protein-protein interaction information as features, and MuSyC parameters as labels, each of the inferred compound representations is combined with the cell line representation and passed through a fully connected neural network (“single compound processor”) to output the parameters describing single compound activity ((C, h, and E). The representations of two compounds are then combined along with the cell line representation to predict interactive parameters (α₁₂, α₂₁, γ₁₂ and γ₂₁) via a fully connected neural network (“combo processor”). These 12 parameters comprise the MuSyC representation and form the last hidden layer of the model and, instead of becoming the primary output of the model or used directly for supervision, these dose response surface model parameters are used to calculate the dose-specific effects of a combination for a given cell line. As a result, the 2D dose response function effectively organizes the last hidden layer in a biochemically meaningful way, such that each number in the last hidden layer is a parameter in the MuSyC function that denotes properties of the combination such as the individual drug EC50 (C₁/C₂), combination cooperativity, and maximum effect (E₃).

In some implementations, a Bayesian framework can be used with an overdispersed likelihood distribution (e.g., a student-t distribution) that compares the measured experimental values to the dose response surface at a given set of MuSyC parameter values. The MuSyC parameter values are further governed by a choice of prior distributions, which can be specified independently per parameter or jointly across parameters. The prior distributions serve to regularize the inferred parameter values, so that the posterior distributions of the Bayesian models cover physically realistic parameter ranges even when experimental data is noisy, sparse, or incomplete. This model can accommodate technical replicates of experimental data (multiple, discrepant measurements at the same dose values) and propagate the sampling uncertainty from this data to the variance of the posterior distribution, thereby accounting for experimental uncertainty.

As an example implementation, prior information can be imposed on the last latent embedding of the model by adding a penalty term to the loss. Specifically, a Gaussian prior distribution can be defined for each MuSyC parameter. During training, the predicted value from the latent embedding which corresponds to each MuSyC parameter (m) is compared to the mean (μ) and standard deviation (σ) of the chosen prior distribution and the penalty (L(m)) is added to the loss, as follows:

$$m \sim N ( μ , σ ) \qquad ( 1 )$$

$$L ( m ) = log ( \frac{1}{\sqrt{2 π} σ} ) * \frac{{( m - μ )}^{2}}{2 {σ}^{2}} \qquad ( 2 )$$

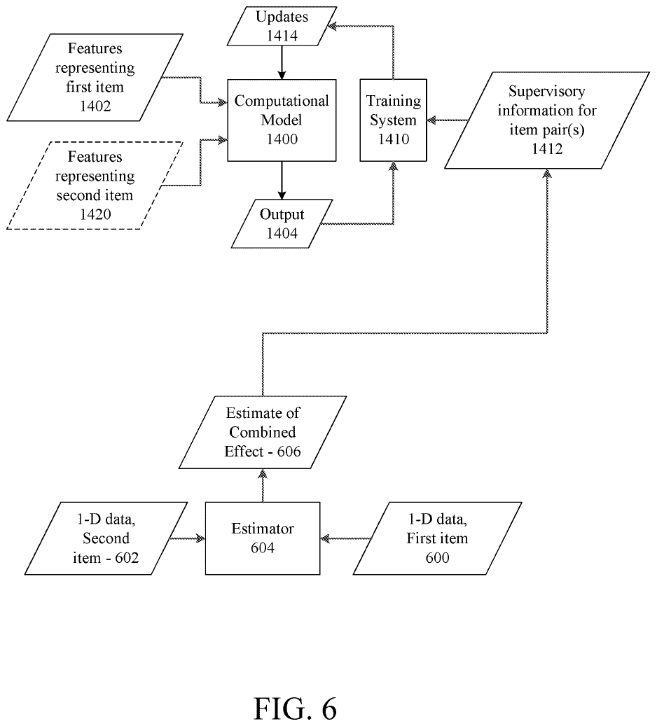

Turning now to FIG. 6, the computational model is similar to the computational model shown in FIG. 4. However, in FIG. 6, this example implementation illustrates how one-dimensional data for two items can be combined to provide supervisory information that is two-dimensional (or multi-dimensional).

For a first researched item, the system has access to a first set of one-dimensional data (600) representing an effect produced by that item in a first quantity. The system also has access to, for a second researched item, a second set of one-dimensional data (602) representing an effect produced by that second researched item in a second quantity. An estimator (604) produces an estimate (606) of the combined effect of the first and second researched items based on data 600 and 602, and a model of interaction of these kinds of items.

For example, many biological responses are in the form of a Hill curve, and combined responses typically are in the form of a 2D Hill surface. Thus, a mathematical representation of a 2D Hill surface models the interaction of compounds that produce a biological response, and the data for a first and second researched item can be fit using this mathematical representation to match a 2D Hill surface. Other models of interaction can be used for researched items in other contexts.

Given the estimate 606 of the combined effect, this estimate can be used as supervisory information 1412 in the context of training a computational model, such as in FIG. 5.

In some implementations, it is helpful to incorporate uncertainty modeling into the computational model. Uncertainty modeling relates to discounting predicted activity of a primary model by, for example, predictions of a secondary model or through specialized post-processing of the predictions of the primary model. The secondary model can be any uncertainty model that can assess the reliability of the primary model. As an example, an uncertainty model can assess differential reliability of a deep neural network (DNN). As another example, an uncertainty model can be an analytical approximation of the uncertainty of the primary model.

An uncertainty model can be in itself a computational model that outputs its own prediction value. The input features for the uncertainty model can be derived in several ways, such as one or more of the following techniques. For example, the input features can be generated using various embedding techniques, such as autoencoders or other transforms, based on the data about the items processed by the primary model. The input features may include the output predictions of the primary model. The input features can include all or a subset of the input features of the primary model. Herein the prediction value output by the uncertainty model is called the “uncertainty value” to distinguish it from the prediction value output by the primary model of which reliability is being assessed. In some implementations it is desirable to assess the suitability of the uncertainty model.

In the context of computational models that predict combined effects, the impact of experimental errors in data in a training set can be significant. For example, there may be multiple measurements of an effect produced by a quantity of a researched item, and such measurements may be made for multiple quantities of the researched item. Instead of averaging multiple measurements into a single measurement representative of the effect of a given quantity of the researched item, all of the measurements can be used during training of the computational model to build an uncertainty model.

Combinatoric laboratory experiments also can be associated with higher experimental sampling uncertainties, which translate to greater predictive uncertainties. In particular, the two-dimensional dose response matrix is sampled experimentally at some fixed number N of points. The sampling precision grows as N\hat{ }2, but the actual number of experimental values is generally fairly low. Also, the observed variability at a fixed dose pair can be quite high, in part because the experimental variation associated with the response to each compound is compounded by the combination.

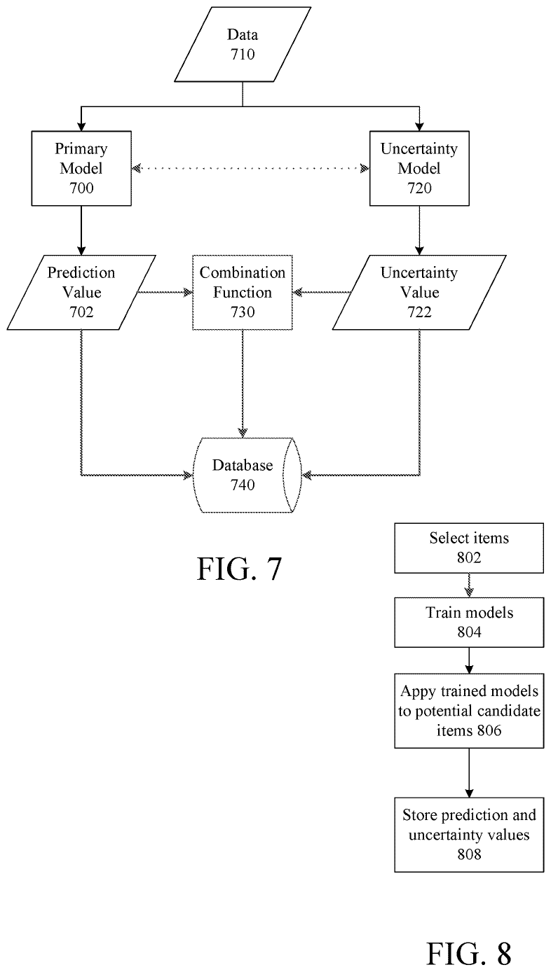

Thus, as shown in FIG. 7, in an example implementation of a computational model incorporating uncertainty modeling, a primary model 700 is the computation model (e.g., model 1400 in FIGS. 4 and 6) that generates the primary prediction values 702 for a combination of items, using data 710. Note, the illustration in FIG. 7 assumes the primary model and uncertainty model already have been trained. For each prediction, the uncertainty model 720 generates an uncertainty value 722. The uncertainty value also can be stored in the database 740 of results (e.g., table 204 in FIG. 2) along with the prediction value 702. A combination function 730 implements one or more functions that combine the prediction value and the uncertainty value. The result of this combination function also can be stored in the database 740 or computed in real time when requested. One or more combination functions can be used, and storage of the prediction value and uncertainty value in the database allows different combination functions to be applied at different times and for different purposes.

Examples of such an uncertainty model include, but are not limited to, the following. One or more uncertainty models, including models of diverse types, can be used in combination.

As one example, a deep ensemble-based model measures variance across an ensemble of primary models, each of which is trained with different random seeds and data subsets. An example of a model is described in “Simple and Scalable Predictive Uncertainty Estimation,” by Ralaji Lakshminarayanan, et al., available at arXiv: 1612.01474v3. Another example of a model is described in “Evaluating Scalable Uncertainty Estimation Methods for DNN-based Molecular Property Prediction,” by Gabriele Scalia et al., and available at arXiv: 1910.03127.

Another technique that can be used is called Bayesian hierarchical stacking. This is a probabilistic method for ensembling predictions of individual deep learning model instances for combinatorics. The result is a distribution of posterior values for each combinatoric prediction generated (e.g., for each MuSyC parameter and for each cell of the checkerboard), even though the individual deep learning models are not probabilistic. Example implementations are described in, for example: 1. Feng, Ji, et al., “Soft Gradient Boosting Machine”, available at https://arxiv.org/pdf/2006.04059.pdf; 2. Sensoy, Murat, et al., “Evidential Deep Learning to Quantify Classification Uncertainty”, in Proceedings of 32 Conference on Neural Information Processing Systems, 2018; and 3. Wenzel, Florian, et al., “Hyperparameter Ensembles for Robustness and Uncertainty Quantification”, available at https://arxiv.org/pdf/2006.13570v2.pdf, 4. Kaplan, David, “On the Quantification of Model Uncertainty: A Bayesian Perspective”, in Psychometrica, Vol. 86, No. 1, pp. 215-238, March 2021, 4. Yuling Yao, et al., “Bayesian hierarchical stacking: Some models are (somewhere) useful”, available at https://arxiv.org/abs/2101.08954, 20 May 2021. In some implementations, ensembling methods may be used, which may be non-Bayesian or non-hierarchical stacking techniques, or both, such as described in Wolpert, David H. “Stacked generalization.” Neural networks 5.2 (1992): 241-259.

In such implementations, both the uncertainty value(s) for an item for a predicted combined effect, as output by the uncertainty model(s), and the predicted combined effect for the item, as output of the computational model, can be used to evaluate predicted candidate items. For example, the uncertainty value for an item as output by the uncertainty model can be combined with the prediction value for the time and the type of combined effect.

In some implementations, a function based on a sum of the uncertainty value and the prediction value can be computed, effectively representing an upper confidence bound. In some implementations, a function based on subtracting the uncertainty value from the prediction value can be computed, effectively representing a lower confidence bound. Weights can be applied to uncertainty values in such functions. In ensembles of primary models, multiple independent uncertainty estimates also can be used in combination.

FIG. 8 is a flowchart describing operation of a computational model incorporating uncertainty modeling. This process is applied to a specified computational model, with a specified training set to be used to train the computational model, and a specified set of potential candidate items to which the trained computational model will be applied, as identified at 802. The specification of the computational model can include a specification of the primary model and the uncertainty model. There can be more than one uncertainty model. The combination function used to combine the prediction values and the uncertainty values also may be specified, or may be specified and applied at a later time.

At 804, the computational model and the uncertainty model are trained using a machine learning algorithm and the training set. This includes training the primary model using the specified training set and training the uncertainty model using the specified training set along with any auxiliary tasks, embeddings, autoencoders, or augmented data.

At 806, the trained models, both the primary model and the uncertainty model(s), are then applied to the set of potential candidate items. The trained primary model outputs a prediction value for each potential candidate item and the type of combined effect. The trained uncertainty model outputs an uncertainty value for each potential candidate item and type of combined effect. These values can be stored (808) in the database, e.g., in table 214. In some implementations, a combination of the prediction value and the uncertainty value can be used to determine whether an item should be identified as a predicted candidate item for which the values should be stored in the database.

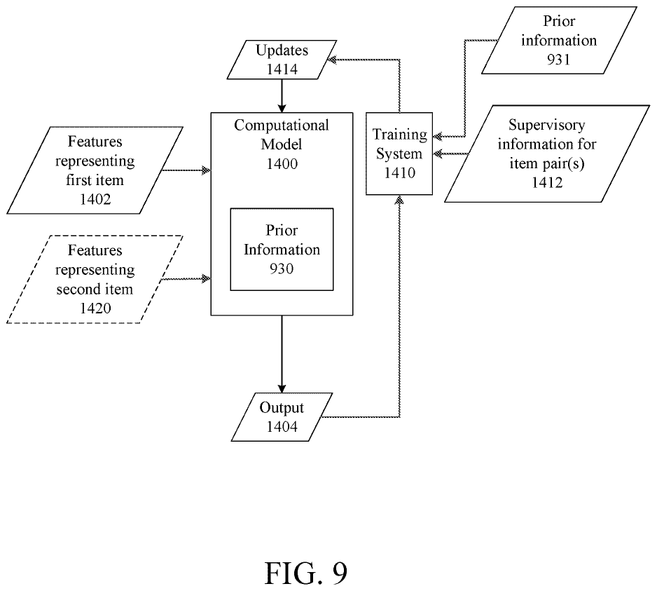

Turning now to FIG. 9, the potential problem of unrealistic predictions will now be addressed. In some implementations, prior information, such as data available for researched items, can be used to ensure that the computational model outputs predicted effects within a physically likely range. The prior information can be data representing a predetermined likely distribution of the combined effect. The prior information can be data representing upper or lower bounds, or both, for the combined effect. Such prior information can be used during training or during inference.

In FIG. 9, the components of the machine learning system are similar to those found in FIGS. 4 and 6 and 11. In some implementations, the computational model 1400 can include prior information 930 as a form of limiter on the output 1404 of the model. For example, it may be known that the combined effect, which is related to the first and second items, cannot be larger than some maximum value, or cannot be smaller than some minimum value, or both. A final stage of the output of the computational model can be to limit the output value to one or both of such values.

In some implementations, prior information 931 can be used by the training system 1410 to adjust the parameters of the computational model. In such implementations, the prior information acts as a form of supervisory information. The output 1404 can be compared to the prior information 931 to compute an error that the training algorithm can minimize.

For example, when fixed upper or lower limits are applied as constraints, they can be integrated through either parameter transformations or asymptotic penalties. The parameter transformations convert untransformed parameters, modeled in an unbounded real number space, to a bounded space (e.g., positive definite) using e.g., an asymptotically logarithmic transformation function.

In some cases, the prior information may be a distribution of likely values for a parameter output by the synergy model (e.g., MuSyC parameters). This distributional prior information is integrated as a regularizing penalty function in the model's loss function during training. This allows the model fitting algorithm to explore any real valued number for the parameter, but penalizes samples according to their consistency with the prior distribution.

In some cases, the conversion module applies prior information about the functional form of the response surface. For example, by choosing a two-dimensional logistic response function, the prior knowledge that the output variables are bounded (i.e. [0-1]) and monotonically increasing is encoded. This choice of conversion function regularizes and constrains the family of output models producible by the framework.

This approach integrates the Bayesian conception of domain-expert designed prior information on latent variables directly into a deep learning modeling framework that seeks to objectively optimize a multi-layer network of weights. This is possible because of the integration of the physically-motivated parameters output by the synergy model as an intermediate output of the deep learning model.

The following explains an example implementation for generating suitable prior distributions. In this example, a hierarchical Bayesian modeling methodology using a Hamiltonian Monte Carlo sampling technique (Hoffman, M. D., et al., “The no-u-turn sampler: adaptively setting path lengths in Hamiltonian Monte Carlo”, in J. Mach. Learn. Res., 15(1): 1593-1623, 2014) is used to establish the posterior distribution of the ground truth dose response surface parameters for compound combinations. The 2D dose response surface function of the MuSyC model can be implemented in the Stan modeling language (Carpenter, B., et al., “Stan: A Probabilistic Programming Language,” in Journal of Statistical Software, 76(1), 1-32, 2017, hereinafter, “Carpenter 2017”). A Gaussian likelihood function can be applied for the model that compares this parameterized dose response function together with a heteroskedastic variance parameter that is a function of the dose grid, as follows:

$${E}_{c 1 , c 2 , l , r} ( d 1 , d 2 ) \sim N ( MuSyC ( Ψ , d 1 , d 2 ) , σ ( d 1 , d 2 ) )$$

In the above equation, E is the experimental measurement of a single biological activity measurement corresponding to one combination of compounds c1 and c2, for one cell line l, for one experimental readout r, at one dose pair d1 and d2; N represents the Gaussian distribution; MuSyC is the MuSyC dose response surface model; Ψ is the vector of 12 biophysical parameters that specifies the dose response surface; and σ is the variance parameter. A step-function is used in the form of σ that reduces the variance allowable for single-compound samples (where either d1 or d2=0) by a factor of 9, reflecting the lower error that typically accompanies the less-experimentally-complex single compound measurements.

The following set of priors for the MuSyC parameters Y′ that reflect biophysical domain knowledge about combination dose responses surfaces were derived:

$${σ}_{0} \sim N ( 0 , 0.1 )$$

$$E 3 \sim N ( 0 , 1 )$$

$$h 1 \& h 2 \sim N ( 0 , 5 )$$

$$log ( {α}_{12} ) \& log ( {α}_{21} ) \sim N ( 0 , 1 )$$

$$log ( {γ}_{12} ) \& log ( {γ}_{21} ) \sim N ( 0 , 1 )$$

The E0 parameter was fixed at identically 1, reflecting the normalization that untreated cells have no drug-induced cytotoxicity. A multivariate prior was used on the C1/C2 and E1/E2 parameters, for which the 2D dose response function induces strong posterior covariance:

$$log ( C 1 ) , E 1 \sim N ( [ \begin{matrix}log ( 100 ) \\ 0\end{matrix} ] , [ \begin{matrix}log ( 100 ) & - 0.75 \\ - 0.75 & 1\end{matrix} ] )$$

These prior distributions can be built and iterated over time based on observations about the goodness of posterior fits. In particular, informative priors can be helpful to regularize parameters that are not well-identified by the experimental data, such as slope parameters that are effectively unobserved when a compound is ineffective within a given cell line.

When using two or more computational models that predict combined effects, it is possible to combine the outputs of the models using a form of ensembling operation. For example, in some implementations the ensembling operation can compute an average of the matrices output by the models for a given pair of items. In some implementations a form of Bayesian stacking can be used. In some implementations, the matrices output by the models can be weighted before ensembling, based on one or more pieces of information. For example, weighting can be based on similarity metrics between the reference items in the training set and the potential candidate items. As another example, weighting can be based on uncertainty metrics output from uncertainty models associated with the predictions.

For example, the Bayesian hierarchical stacking technique averages model instances in an ensemble where a different weight vector across model instances is generated for each sample. In particular, the weight for each model instance is predicted as a function of covariant parameters. For example, if the sample is a combination of compounds, the covariates can specify the chemical class or structural properties of each compound or the union thereof. The model is trained using samples with ground truth outcome labels and then can predict optimal model weights applicable to novel items.

When using two or more computational models that predict combined effects, additional data can be provided along with the data representing the predicted combined effects, to provide more insights into the predictions.

For example, a response score can be computed based on a predicted effect at a specific combination of quantities of the items, e.g., maximum tolerable dose on a targeted cell line. As another example, a response score can be computed based on a quantity-independent measure of relative synergy, such as any value that quantifies synergistic efficacy (an example of which is described below).

As another example a potential score can be computed based on a summary parameter of the combinatoric effects across all items for which a response of a particular type is predicted. This potential score represents a generalized combinatoric potential based on a relative synergy score averaged over multiple combinations of quantities of the items, and thus is not a quantity-specific prediction.

As another example, an ensemble score can be provided, as a weighted average of other scores such as a weighted average of the response score and the potential score.

As an example implementation of a response score, given a specific combination of quantities of items, a respective predicted combined effect from each of a plurality of models for this specific combination is obtained. A function of these respective predicted combined effects is computed, such as an average or other function. This response score can be normalized.

As an example implementation of a potential score, the data representing the respective predicted combined effects from each of a plurality of models is processed to generate a first value that quantifies synergistic efficacy, and a second value, which quantifies the combined effect at the maximum quantities of both items. Any technique for quantifying synergistic efficacy can be used, such as a function that is based on a. the combined effect at the maximum quantities of both items, b. the effect of the maximum quantity of one of the items, c. the combined effect at the minimum quantities of both items, and d. the effect of the minimum quantity of one of the items. A function of the first and second values, such as their product, can be averaged over the number of models. This potential score can be normalized.

As an example implementation of an ensemble score, a weighted average of the form of a weight (Wr) applied the response score, and a weight (Wp) applied to the potential score, divided by the sum of the weights (Wr+Wp) can be used. The response score and potential score can be normalized prior to weighting.

Examples of such data are shown in Table II below.

TABLE II

| Term | Description | Source |
|---|---|---|
| One or more parameters | One or more parameters | Output from a |
| representing combined effect | representing the combined effect | computational |
| | | model |
| Response Score | Based on the specific predicted | Derived from |
| | effect at a specific combination of | parameters |
| | quantities of the items, e.g., doses on | representing |
| | a particular cell line | combined effect |
| Potential Score | based on the overall ‘potential’ for | Derived from |
| | synergy based on averaging a | parameters |
| | summary parameter of combinatoric | representing |
| | effects across all predicted items | combined effect |
| Ensemble Score | A weighted average of the response | Derived from |
| | score and the potential score. | parameters |
| | | representing |
| | | combined effect |

In some implementations, it is desirable to include a mechanism to ensure that the predictive model produces the same results whether the items are presented as the combination of “item 1” and “item 2” or as the combination of “item 2” and “item 1”, if the ordering of the items in the combination does not matter. One mechanism that can be used is to tie model weights as applied to features of the first and second items together. Therefore, in such implementations, all the weights and layers within the computational model are shared between the two items. An example of this technique is shown in Xia, Fangfang, et al., “Predicting tumor cell line response to drug pairs with deep learning”, BMC Bioinformatics, 19(Supp. 18): 486 (2018).

Such machine learning models that predict combined effects have several practical uses. Some applications relate to predicting the combined effects of one or more candidate items together on a property of a target. Typically, such applications involve a multidimensional measure of performance in response to two or more inputs, where the measure of performance can be represented as a response surface over the domain of the two or more inputs.

Examples of such applications include but are not limited to predicting drug interactions, food-drug interactions, and effects of combinations of food compounds.

As a specific example of an application, such combinatoric modeling can be used to understand combination therapies. As an example, combination therapy is an effective cancer treatment strategy because it has been shown, in optimal cases, to reduce drug resistance and toxicity. However, the possibility space of therapeutic combinations is immense, and it is laborious and expensive to screen all possible drug pairs. This potential space is even larger when considering variable dosage. Computational approaches such as machine learning models can help prioritize effective drug combinations and accelerate screening efforts. A machine learning model as described herein can predict the dose response surface of drug combinations to better describe the interactive effects of drug combinations. More specifically, it can predict the dose response surface of a two-drug combination based on each drug's known protein target interactions. In one implementation, a graph neural network (GNN) can be used to encode a family of 2D logistic response surfaces as a latent parameterization. The model can be trained using public combinatoric anti-cancer screening datasets such as that published by O'Neil et al, “An unbiased oncology compound screen to identify novel combination strategies,” Mol. Cancer Ther. 2016; 15:1155-1162, or as available as described in Zagidullin, Bulat, et al. “DrugComb: an integrative cancer drug combination data portal”, in Nucleic Acids Research, 2019, Vol. 47, Web Server issue W43-W51. Using the protein targets of the compounds and protein-protein interaction network as inputs, the GNN can be applied to infer pathways perturbed by the compounds most predictive of cellular effects.

In example implementation of such a model was trained using the O'Neil at al. dataset. This dataset provides drug combination cell viability screening data and was produced by screening combinations of 38 oncology drugs on 39 cancer cell lines on a four-by-four dose matrix with four replicates per dose combination, producing a total dataset size of 22,000 dose response surfaces (experimental “checkerboards”) and 1,475,332 individual cell viability measurements. Each drug combination was normalized to the untreated condition to obtain fractional viability at each dose combination, which was used to fit the MuSyC parameters or directly train the model. In order to ensure symmetry during model training, the training set was augmented by flipping the order of the drug combination; this doubles the training set size and helps ensure equivalent predictions regardless of drug order. To test the performance of the model on novel drug combinations, a ten-fold cross validation set was created by leaving out drug pairs from the training data.

While this data, and the model using it, is based on a public oncology dataset, the model architecture is suitable to discover novel combinations in other disease areas such as immunological, infectious, and metabolic diseases. Even if experimental, ground truth combination data is limited, the model architecture can be applied because of the data efficiency and regularizing effect of the model.

The model is data efficient as it can incorporate data points from any position on the dose response surface. This versatile format enables data to be integrated from across experiments. For example, public high throughput screening (HTS) datasets for single compound libraries can be used by integrating them with a smaller combinatoric screening data in the same assay, or by combining initial primary drug screens (typically done at fixed dose) and confirmatory screens (sampling multiple doses in the same assay) for single compound experiments, or for combinatorics experiments, or both. Furthermore, classical synergy prediction approaches collapse information across dose samples and replicates onto one data point (e.g. a ZIP score at a nominal dose). If an additional experiment were performed testing a compound combination at higher doses, it would not alter the ZIP score at the nominal dose and would be discarded from the modeling process. In our model, however, this additional sample would help supervise the dose response surface parameters, particularly E₃, and may therefore improve dose response surface reconstruction overall. Proposed drug combinations can be prioritized and nominated for further evaluation using any metric (ZIP, Lowe, CI at any dose, or MuSyC β).

The models also have an additional regularizing effect suitable for small datasets. The adoption of the parametric form of the dose response surface prevents the prediction of unphysical dose response surface shapes. For example, dose “hotspots” that may appear due to aleatoric noise in experimental data that make one dose combination appear significantly more effective than neighboring doses are smoothed out in fitted models generated by the techniques herein. While there is some risk of model misspecification, a response shape resembling a 2D generalization of the Hill curve is widely observed in combinatoric drug screening. The model is additionally trained with experimental replicate-level data, effectively supervising with the actual measurement label distribution, which provides implicit regularization.

Such a model can predict the dose response surfaces of drug combinations. Excellent dose response surface reconstruction was observed for drugs whose individual dose response profiles are known. This experiment demonstrated that dose response surface prediction of de novo combinations is possible with significant performance improvement above baseline, which supports in silico prediction of the therapeutic window and better understanding of drug interaction effects. More accurate reconstruction of de novo combination dose response curves could be obtained with increased availability of training data across chemical diversity. To test if the latent parameters inferred by the model directly from dose-response surfaces are biophysical, the latent parameters were compared to parameters individually fitted to each 2D response surface. Imposing a Bayesian prior via L2 regularization helps the model infer latent parameters more consistent with biochemical intuition. The distribution of the inferred parameters reflects the relatively low interactive potential of screened ground truth drug pairs, consistent with previous experimental observation. Thus, a biophysically-motivated model with a regularizing Bayesian prior can have high predictive power in reconstructing 2D dose response surfaces for drug combinations.

While the model presented here is based on a two-dimensional dose response curve to predict drug combination effects, this parametric approach can be easily generalized to predict the activation/inhibition effect on a protein target or other dose response of K>2 (multiple compound) combinations.

This latent parameterization approach can also be extended to areas such as material sciences and formulation development where observables such as time, particle size, impurity and composition, temperature, and other physical or chemical factors can be described by a multi-dimensional parameterized mathematical model. Another example application is predicting power-performance behavior of a system, such as for various computing, electrical, mechanical, and power generation systems. For example, different scheduling algorithms for multiple processes executed on a processor may result in different power consumption and performance for that processor when measured as a function of two configuration variables of the algorithm.

Another example application is in predicting performance of materials, such as alloys or other materials. For example, the doping of a material with multiple dopants at different concentrations may result in different electrical properties. As another example, tensile strength or other property of an alloy may be different from different concentrations of its various component metals or additives.

Another example relates application to optimizing properties of a pharmaceutical formulation, or other product of chemical manufacture. For example, for different settings for 1) a temperature or other experimental condition during a step of formulation and 2) a particle size or other property of a component of an emulsion, the bioavailability or other property of the resulting product may change.

Another example application is hyperparameter optimization for a machine learning model. For example, with different numbers of base learners in an ensemble and different learning rates, the held out predictive performance may change.

Another example application is analyzing consumer price sensitivity. With different costs and different packaged quantities of a product, such as cereal, consumer demand may vary.

Another example application is analyzing digital advertising response. For example, with different durations for an advertisement and different average ages of a targeted audience, engagement time for the advertisement may change. As another example, A/B testing can be extended to multiple variables for which the combined effects can be analyzed.

By making such predictions, laboratory experiments can be performed to validate the predictions. For example, an assay can be performed with a candidate compound and a selected protein to characterize the interaction of the candidate compound and the selected protein. Interaction information for a plurality of compounds can be aggregated. This aggregated information can be used to characterize an overall effect of the plurality of compounds with respect to a health condition or activity of a drug.

The foregoing description provides example implementations of a computer system implementing these techniques. The various computers used in this computer system can be implemented using one or more general-purpose computers, such as client devices including mobile devices and client computers, one or more server computers, or one or more database computers, or combinations of any two or more of these, which can be programmed to implement the functionality such as described in the example implementations.

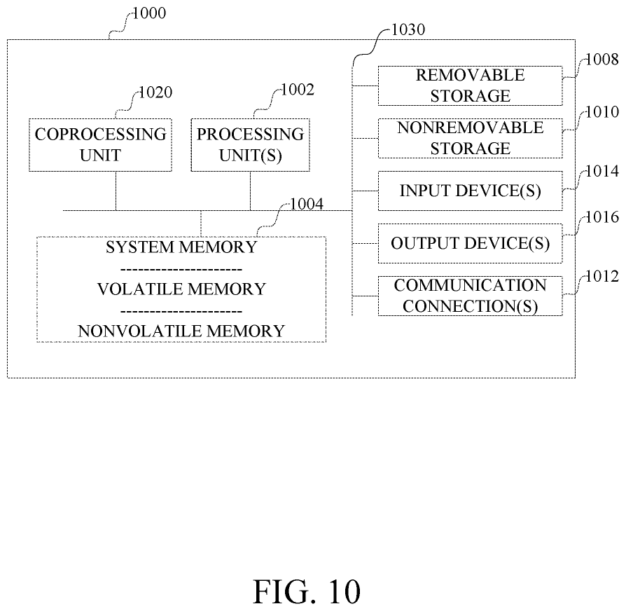

FIG. 10 is a block diagram of a general-purpose computer which processes computer programs using a processing system. Computer programs on a general-purpose computer generally include an operating system and applications. The operating system is a computer program running on the computer that manages access to resources of the computer by the applications and the operating system. The resources generally include memory, storage, communication interfaces, input devices and output devices.

Examples of such general-purpose computers include, but are not limited to, larger computer systems such as server computers, database computers, desktop computers, laptop and notebook computers, as well as mobile or handheld computing devices, such as a tablet computer, handheld computer, smart phone, media player, personal data assistant, audio and/or video recorder, or wearable computing device.

With reference to FIG. 10, an example computer 1000 comprises a processing system including at least one processing unit 1002 and a memory 1004. The computer can have multiple processing units 1002 and multiple devices implementing the memory 1004. A processing unit 1002 can include one or more processing cores (not shown) that operate independently of each other. Additional coprocessing units 1020, such as a graphics processing unit or field programmable gate array or other processor, also can be present in the computer. The memory 1004 may include volatile devices (such as dynamic random-access memory (DRAM) or other random-access memory device), and non-volatile devices (such as a read-only memory, flash memory, and the like) or some combination of the two, and optionally including any memory available in a processing device. Other memory, such as dedicated memory or registers also can reside in a processing unit. Such a memory configures is delineated by the dashed line 1004 in FIG. 10. The computer 1000 may include additional storage (removable and/or non-removable) including, but not limited to, solid state devices, or magnetically recorded or optically recorded disks or tape. Such additional storage is illustrated in FIG. 10 by removable storage 1008 and non-removable storage 1010. The various components in FIG. 10 are generally interconnected by an interconnection mechanism, such as one or more buses 1030.

A computer storage medium is any medium in which data can be stored in and retrieved from addressable physical storage locations by the computer. Computer storage media includes volatile and nonvolatile memory devices, and removable and non-removable storage devices. Memory 1004, removable storage 1008 and non-removable storage 1010 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 1000 may also include communications connection(s) 1012 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals. Communications connections 1012 are devices, such as a network interface or radio transmitter, that interface with the communication media to transmit data over and receive data from signals propagated through communication media.

The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 1000 may have various input device(s) 1014 such as a various pointer (whether single pointer or multi-pointer) devices, such as a mouse, tablet and pen, touchpad and other touch-based input devices, stylus, image input devices, such as still and motion cameras, audio input devices, such as a microphone. The computer may have various output device(s) 1016 such as a display, speakers, printers, and so on, also may be included. These devices are well known in the art and need not be discussed at length here.

The various storage 1010, communication connections 1012, output devices 1016 and input devices 1014 can be integrated within a housing of the computer, or can be connected through various input/output interface devices on the computer, in which case the reference numbers 1010, 1012, 1014 and 1016 can indicate either the interface for connection to a device or the device itself as the case may be.

An operating system of the computer typically includes computer programs, commonly called drivers, which manage access to the various storage 1010, communication connections 1012, output devices 1016 and input devices 1014. Such access generally includes managing inputs from and outputs to these devices. In the case of communication connections, the operating system also may include one or more computer programs for implementing communication protocols used to communicate information between computers and devices through the communication connections 1012.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system.

Each component (which also may be called a “module” or “engine” or “computational model” or the like), of a computer system such as described herein, and which operates on one or more computers, can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

In some implementations, computer program instructions for performing statistical processing can be implemented using the STAN statistical programming package, such as described in: Stan Development Team (2017b). Stan Modeling Language: User's Guide and Reference Manual. URL http://mc-stan.org/manual.html, and Carpenter 2017.

Such a system can be implemented in conjunction with a system as described in PCT Publication 2022/240751, and U.S. Provisional Patent Application Ser. No. 63/386,161, filed Jul. 12, 2022, entitled “PROCESSES, MACHINES, AND ARTICLES OF MANUFACTURE RELATED TO PREDICTING EFFECTS OF COMBINATIONS OF ITEMS”.

Each reference, e.g., non-patent publications, patents, and patent applications, cited herein is hereby expressly incorporated by reference herein in its entirety. In the event of conflict between subject matter herein and subject matter in such a reference, the subject matter herein controls.

It should be understood that the subject matter defined in the appended claims is not necessarily limited to the specific implementations described above. The specific implementations described above are disclosed as examples only.

What is claimed is:

1. A computer system for predicting properties of combinations of items, comprising:
a processing system configured to:
train a computational model using data representing researched items,
wherein the data representing researched items includes, for each pair of researched items, respective quantitative information describing a combined effect of the pair of researched items together in a plurality of different combinations of quantities,
wherein the respective quantitative information for each pair of researched items comprises a respective set of values describing a response manifold; and
input data representing a plurality of potential candidate items to the trained computational model such that the trained computational model outputs a result set,
wherein the result set includes respective predicted information, for each predicted candidate item from among the plurality of potential candidate items, describing a combined effect of at least the predicted candidate item and another item together in a plurality of different combinations of quantities,
wherein the respective predicted information for each predicted candidate item comprises a respective set of values describing an N-dimensional variable slope sigmoidal manifold, wherein N is a positive integer greater than or equal to two.

2. The computer system of claim 1, wherein the inputs of the computational model further comprises inputs to receive second data representing the second item.

3. The computer system of claim 1, wherein the quantitative information comprising the set of values describing the response manifold describing the combined effect comprises a respective matrix of values.

4. The computer system of claim 1, wherein the quantitative information comprising the set of values describing the response manifold describing the combined effect comprises a respective set of values for a set of parameters of a model describing an M-dimensional variable slope sigmoidal manifold.

5. The computer system of claim 1, wherein the predicted information comprising the set of values describing the response manifold describing the combined effect comprises a respective set of values for a set of parameters of a model describing an N-dimensional variable slope sigmoidal manifold.

6. The computer system of claim 1, wherein the predicted information comprising the set of values describing the response manifold describing the combined effect comprises a respective matrix of values.

7. The computer system of claim 1, wherein the computational model includes parameters representing uncertainty associated with measurements of the combined effect, and wherein the respective predicted information for each predicted candidate item comprises a respective set of values for the parameters representing uncertainty associated with measurements of the combined effect.

8. The computer system of claim 1, wherein the set of values describing the response manifold is fitted probabilistically.

9. The computer system of claim 1, wherein supervisory information for training the computational model comprises, for each pair of researched items, a multidimensional representation of the respective combined effect of the pair of researched items together for the plurality of different combinations of quantities.

10. The computer system of claim 9, wherein the multidimensional representation of the respective combined effect of the pair of researched items together for the plurality of different combinations of quantities comprises a respective matrix of values.

11. The computer system of claim 9, wherein the multidimensional representation of the respective combined effect of the pair of researched items together for the plurality of different combinations of quantities comprises a respective set of values for a set of parameters of a model describing an M-dimensional variable slope sigmoidal manifold, wherein M is a positive integer greater than or equal to two.

12. The computer system of claim 9, wherein the multidimensional representation of the respective combined effect of the pair of researched items together for the plurality of different combinations of quantities comprises a respective estimate for the quantitative information describing the combined effect of the pair of researched items together in a plurality of different combinations of quantities based on known data for at least one of the researched items.

13. The computer system of claim 1, wherein the respective predicted information describing the combined effect is maintained within a physically likely range based on data representing a predetermined likely distribution of the combined effect.

14. The computer system of claim 1, wherein the data representing researched items includes quantitative information describing a first effect of the first item in a first plurality of quantities and quantitative information describing a second effect of the second item in a second plurality of quantities, and wherein the generating the initial estimate of the quantitative information describing the combined effect is based on an estimate of a matrix of values based on the quantitative information describing a first effect of the first item in a first plurality of quantities and quantitative information describing a second effect of the second item in a second plurality of quantities.

15. The computer system of claim 1, wherein the M-dimensional variable slope sigmoidal manifold comprises a sigmoidal surface.

16. The computer system of claim 1, wherein the M-dimensional variable slope sigmoidal manifold is defined by one of:
a. a two-dimensional Hill function; and
b. a combination of a first one-dimensional variable slope sigmoidal curve and a second one-dimensional variable slope sigmoidal curve.

17. The computer system of claim 1, wherein the M-dimensional variable slope sigmoidal manifold represents a combination of a first dose-response curve for the first item and a second dose-response curve for the second item.
